SEQUENCE LISTING (3 sequences)

SEQ ID NO: 1 - DNA, 1482 bp, Homo sapiens
gcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaatta

SEQ ID NO: 2 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 3 - DNA, 880 bp, Artificial Sequence
  Description of Artificial Sequence Synthetic polynucleotide
ggccatgtcgagtgggggcagtggagggggagtgccagaacaggaagattccgtgctgttcaggcgaggaaccgggcagagtgacgacagtgacatttgggacgacacggccctgatcaaggcctatgacaaagccgtggcctccttcaagcacgcgctgaagaacggcgacatttgcgaaaccagcggcaagcctaagaccacccctaaacggaagcccgccaagaaaaataagtcccagaaaaagaacacagccgcaagtcttcagcaatggaaggtgggggataagtgctccgcgatatggagtgaagacgggtgcatctatcctgccaccatcgccagcatagacttcaagcgcgaaacctgcgtggtggtgtacactggatacgggaaccgggaggagcagaacctgagcgacctgttgagccctatttgtgaggtggccaacaacatcgagcagaatgcgcaagaaaatgaaaacgagagtcaggtgtccaccgatgagagtgaaaacagtaggagccccggcaacaaatccgacaatatcaagcccaaaagcgcaccctggaatagcttccttccacccccccccccaatgcccggacctcgactgggccccggaaagcctggcctgaagttcaacggcccccctcctcctcctccccctcctcccccccacctgctgagctgctggttgccccctttcccttcgggaccccctatcatacctccccccccccctatttgccctgactccctggacgacgcggacgcgctgggcagtatgctcatctcgtggtacatgtcaggataccacaccgggtactacatgggcttcagacaaaatcagaaggaaggacgatgtagtcactccctgaat

(19) United States
(12) Patent Application Publication
(10) Pub. No.: US 2022/0267798 A1
(43) Pub. Date: Aug. 25, 2022

(54) POTENCY ASSAYS FOR VIRAL VECTOR PRODUCTION

(71) Applicant: Biogen MA Inc., Cambridge, MA (US)

(72) Inventors: Marina Feschenko, Stoneham, MA (US); Svetlana Bergelson, Newton, MA (US); Anthony Christophe Leyme, Medford, MA (US)

(21) Appl. No.: 17/632,874

(22) PCT Filed: Aug. 7, 2020
(86) PCT No.: PCT/US2020/045423

§ 371 Date: Feb. 4, 2022

Related U.S. Application Data
(60) Provisional application No. 62/884,252, filed on Aug. 8, 2019.

(51) Int. Cl. C12N 15/86; C07K 14/475; G01N 33/50

(52) U.S. Cl. CPC C12N 15/86; C07K 14/475; G01N 33/5023; C12N 2750/14143; C12N 2750/14152; G01N 2333/4703; G01N 2500/10

(57) ABSTRACT

The present disclosure provides sensitive and robust assays for determining the potency of payloads encoded by recombinant viral vectors. Particularly, the present disclosure provides assays to determine the potency of SMN polypeptide as expressed by recombinant viral vectors used for the treatment of spinal muscular atrophy. The present description encompasses, inter alia, methods for determining potency (e.g., biological activity), e.g., relative potency, of a recombinant viral vector.

### CROSS REFERENCE TO RELATED APPLICATIONS

This application claims priority to U.S. Provisional Patent Application No. 62/884,252, filed on Aug. 8, 2019, which is incorporated herein by reference in its entirety.

### BACKGROUND

Viral vector mediated gene therapy is a rapidly developing therapeutic field. Many aspects of therapeutic viral vectors will need to be evaluated to determine safety and efficacy prior to use as a therapy.

Thus, there remains a need for improved methods for determining potency of a recombinant viral vector. In particular, there is a need for methods that allow for improved determination of potency of a payload, such as a polypeptide, expressed by a recombinant viral vector.

### SUMMARY

The present description encompasses, inter alia, methods for determining potency (e.g., biological activity), e.g., relative potency, of a recombinant viral vector. Methods described herein have improved characteristics over prior methods for determining potency of a payload (e.g., a polypeptide, such as a SMN polypeptide) expressed by a recombinant viral vector. In some embodiments, use of modified host cells with decreased expression of at least one payload (e.g., at least one SMN polypeptide) relative to an unmodified reference host cell of the same type allows for improved potency assays of recombinant viral vectors described herein. In some embodiments, modified host cells (e.g., human modified host cells, e.g., SH-SY5Y KD cells) used in methods described herein are more physiologically relevant and/or easier to culture than other types of host cells (e.g., primary cells and/or non-human mammalian cells, e.g., mouse cells). In some embodiments, SH-SY5Y KD cells comprise a knockdown (e.g., constitutive or conditional) in a SMN gene (e.g., a SMN1 or SMN2 gene), e.g., comprising or expressing an inhibitory nucleic acid against a SMN gene (e.g., a SMN1 or SMN2 gene), e.g., a shRNA against SMN1 (e.g., a doxycycline inducible shRNA against a SMN1 gene, e.g., shRNA120 or shRNA 128).

Such improved characteristics can include, but are not limited to: (i) the method can be performed completely without use of a helper function (e.g., Ad2 or Ad5 helper virus); (ii) lower amounts of recombinant viral vector can be required for transduction than transducing with an unmodified reference host cell of the same type or a different type; (iii) the method has a low standard deviation; (iv) potency can be determined with an accuracy of about 90%, about 91%, about 92%, about 93%, about 94%, about 95%, about 96%, about 97%, about 98%, about 99%, or more; (v) potency can be determined with a precision of about 5%, about 6%, about 7%, about 8%, about 9%, or about 10%; (vi) the method can indicate stability of recombinant viral vectors described herein, e.g., following thermal stress of the recombinant viral vector; (vii) potency of recombinant viral vectors described herein is not affected by presence of empty capsids; and/or (viii) able to achieve high signal to noise due to choice of a modified host cell line (e.g., SH-SY5Y KD cells comprising a knockdown (e.g., constitutive or conditional) in a SMN gene (e.g., a SMN1 or SMN2 gene), e.g., comprising or expressing an inhibitory nucleic acid against a SMN gene (e.g., a SMN1 or SMN2 gene), e.g., a shRNA against SMN1 (e.g., a doxycycline inducible shRNA against a SMN1 gene, e.g., shRNA120 or shRNA 128)).

In one aspect, the disclosure provides methods of determining potency of a recombinant viral vector encoding at least one SMN polypeptide comprising: (a) transducing modified host cells with the recombinant viral vector, wherein the modified host cells comprise decreased expression of at least one SMN polypeptide relative to an unmodified reference host cell of the same type; (b) contacting the modified host cells with a first agent for detection of the SMN polypeptide; (c) contacting the modified host cells with a second agent comprising a detection moiety for detection of the first agent; and (d) detecting presence of Gemini of coiled bodies (GEMs), thereby determining potency of at least one SMN polypeptide.

In some embodiments, a recombinant viral vector comprises an adeno-associated viral (AAV) vector, an adenoviral vector, or a retroviral vector. In some embodiments, a retroviral vector comprises a lentiviral vector or a gammaretroviral vector. In some embodiments, an AAV vector comprises AAV1, AAV2, AAV3, AAV4, AAV5, AAV6, AAV7, AAV8, AAV9, AAV10, AAV11, or a variant thereof. In some embodiments, an AAV vector comprises AAVhu68. In some embodiments, an AAV vector comprises a SMN1 gene operably linked to a chicken-β actin promoter (CB7). In some embodiments, an AAV vector comprises two ITRs flanking a SMN1 gene. In some embodiments, an AAV vector comprises a rabbit β-globin polyA signal.

In some embodiments, modified host cells comprise a conditional knockdown or a knockout of an SMN1 gene. In some embodiments, modified host cells comprise at least one shRNA for conditional knockdown of an SMN1 gene. In some embodiments, at least one shRNA: (i) comprises shRNA120 or shRNA 128, and/or (ii) does not target or affect a recombinant viral vector described herein. In some embodiments, modified host cells comprise or are mammalian host cells. In some embodiments, modified host cells comprise or are human cells. In some embodiments, modified host cells comprise or are SH-SY5Y cells. In some embodiments, modified host cells comprise or are SH-SY5Y KD cells.

In some embodiments, prior to transduction, one or more of the following occurs: (i) host cells are frozen and thawed at least once; (ii) modified host cells are passaged at least 3 times; (iii) host cells are treated with doxycycline (e.g., to induce knockdown of a SMN gene); and/or (iv) host cells are seeded at a density of about 5.0×10³ to about 5.0×10⁴ cells/well.

In some embodiments, modified host cells are seeded and transduced within a 24 hour period. In some embodiments, a transduction step is performed at about 5 different MOIs achieved by serial dilution. In some embodiments, a transduction step (b) is performed with an MOI of about 6.1×10⁵ VG/cell to about 4×10⁶ VG/cell (e.g., about 6.1×10⁵ VG/cell, about 9.8×10⁵ VG/cell, about 1.6×10⁶ VG/cell, about 2.5×10⁶ VG/cell, and about 4×10⁶ VG/cell). In some embodiments, a signal to noise ratio is greater than or about 2.5.

In some embodiments, a first agent comprises an anti-SMN1 antibody or an antigen-binding fragment thereof or an aptamer. In some embodiments, a detection moiety comprises or is a fluorescent, colorimetric, or enzymatic label. In some embodiments, a second agent comprises a fluorescently labeled secondary antibody or an antigen-binding fragment thereof. In some embodiments, presence of GEMs is detected by immunofluorescence. In some embodiments, presence of GEMs is detected by imaging. In some embodiments, imaging comprises or is High-Content Imaging (HCI).

In some embodiments, methods described herein are performed without or substantially without use of at least one helper function. In some embodiments, at least one helper function comprises an Ad2 or Ad5 helper virus.

In some embodiments, lower amounts of recombinant viral vector are required for transduction than transducing with an unmodified reference host cells of the same type or a different type. In some embodiments, methods described herein have a low standard deviation.

In some embodiments, potency is determined with an accuracy of about 90%, about 91%, about 92%, about 93%, about 94%, about 95%, about 96%, about 97%, about 98%, about 99%, or more. In some embodiments, potency is determined with a precision of about 5%, about 6%, about 7%, about 8%, about 9%, or about 10%.

In some embodiments, methods described herein indicate stability of a recombinant viral vector, e.g., following thermal stress of a recombinant viral vector. In some embodiments, potency of a recombinant viral vector is not affected by presence of empty capsids. In some embodiments, a recombinant viral vector comprises a plurality of empty virus capsids.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The Figures described below, which together make up the Drawing, are for illustration purposes only, not for limitation.

FIG. 1 shows high variability in GEMs detection in HeLa-RC32 cells. HeLa-RC32 cells were plated per well of a 96-well plate. Cells were seeded 24 hours prior co-infection with a titration curve of rAAVhu68-SMN1 (1×10⁶ to 3.1×10⁴ VG/cell) and human adenovirus 5 at a MOI of 50. Cells were infected for 2 days then fixed and stained for SMN. The number of GEMs/cell was assessed by high-content imaging using the CX5 CellInsight.

FIG. 2 show Knock-Down of SMN1 in SH-SY5Y shRNA120 and SH-SY5Y shRNA 128 DOX treated cells. Untreated cells were trypsinized, centrifuged then seeded at 1×10⁶ cells per 6-well plate or frozen (T=0) at −70° C. The seeded cells were treated with DOX (100 μg/mL) for 3 and 7 days prior harvest. The harvested cells were washed in PBS, centrifuged and the pellet frozen at −70° C. Western blotting was performed according to the manufacturer's protocols using an Odyssey Infrared Imaging System (Li-COR Biosciences). Data quantified from immunoblots was normalized by a loading control: tubulin.

FIG. 3 shows a correlation between cell passages of SH-SY5Y sh120 cells and number of GEMs formed. Cells at passage 11, 15 and 25 (left panel) and cells at passage 4, 7 and 15 (right panel) were treated with DOX (100 μg/mL) for 3 days and frozen. 1×10⁴ thawed cells were plated per well of a TC-treated 96-well plate for 24 hours prior infection. Cells were infected with a titration curve of rAAVhu68-SMN1 (4×10⁶−6.1×10⁵ VG/cell) for 2 days then fixed and stained for SMN. The number of GEMs/cell was detected by high-content imaging using the CX5 CellInsight.

FIG. 4 shows a comparison of cultured and “thaw-and-go” SH-SY5Y sh120 cells. 2×10⁴ in-culture or “thaw-and-go” cells were plated per well of a PDL-coated 96-well plate. Cells were plated 24 hours prior infection with a titration curve of rAAVhu68-SMN1 (2×10⁶−6.25×10⁴ VG/cell). Cells were infected for 2 days and then fixed and stained for SMN. The number of GEMs/cell was assessed by high-content imaging using the CX5 CellInsight.

FIG. 5 shows determination of the optimal cell density of SH-SY5Y sh120 cells. Thawed SH-SY5Y sh120 cells pre-treated with DOX (100 μg/mL) were plated at a density of: (A) 2, 2.5 and 3×10⁴ cells/well, (B) 1.5 and 2×10⁴ cells/well and (C) 1×10⁴ cells/well and infected with either 1:2 (A and B) or 1:1.5 (C) serial dilutions of rAAVhu68-SMN1. Cells were treated for 2 days then fixed and stained for SMN as described in TD-TDMP-990. The number of GEMs/cell was assessed by high-content imaging using the CX5 CellInsight.

FIG. 6 shows an effect of length of infection of SH-SY5Y sh120 cells on assay performance. 2×10⁴ cells/well were plated in a TC-treated 96-well plate 24 hours prior infection with a titration curve of rAAVhu68-SMN1 (2×10⁶−2.1×10⁴ VG/cell). Cells were fixed and stained for SMN after 48 or 72 hours after infection. The number of GEMs/cell was assessed by high-content imaging using the CX5 CellInsight.

FIG. 7 shows same-day versus next-day treatment of SH-SY5Y sh120 cells with rAAVhu68-SMN1. 1×10⁴ cells/well were plated in a TC-treated 96-well plate. Cells were seeded for 6- or 24-hours prior infection with a titration curve of rAAVhu68-SMN1 (4×10⁶−6.1×10⁵ VG/cell). Cells were incubated for 2 days then fixed and stained for SMN. The number of GEMs/cell was assessed by high-content imaging using the CX5 CellInsight.

FIG. 8 shows SH-SY5Y sh120 cell infection with and without human adenovirus 5. 2×10⁴ in-culture cells were plated per well of a PDL-coated 96-well plate. Cells were seeded for 24 hours prior infection with a titration curve of rAAVhu68-SMN1 (1×10⁶−3.1×10⁴ VG/cells)±human adenovirus 5 at a MOI of 50. Cells were infected for 2 days then fixed and stained for SMN. The number of GEMs/cell was assessed by high-content imaging using the CX5 CellInsight.

FIG. 9 shows comparison of SH-SY5Y KD cell fixation at 4° C. and RT. SH-SY5Y sh120 cells pre-treated with DOX (100 μg/mL) were plated at 1×10⁴ cells/well and infected with rAAVhu68-SMN1 at a 1:1.6 serial dilution. Cells were treated for 2 days then fixed in 4% PFA/PBS at 4° C. or RT for 20 minutes prior staining for SMN. The number of GEMs/cell was assessed by high-content imaging using the CX5 CellInsight.

FIG. 10 shows a comparison of blocking buffers. SH-SY5Y KD cells were seeded at a density of 1×10⁴ cells per well in a 96-well plate and infected the following day with rAAVhu68-SMN1 for 2 days. SH-SY5Y KD cells were blocked for 1 hour in LI-COR blocking reagent or 5% NGS/PBS then stained for SMN. The number of GEMs/cell was assessed by high-content imaging using the CX5 CellInsight.

FIG. 11 shows that the potency assay is reproducible. 2×10⁴ in-culture SH-SY5Y KD cells were plated per well of a poly-D-Lysin (PDL)-coated 96-well plate. Cells were seeded for 24 hours prior infection with a titration curve of rAAVhu68-SMN1 (2×10⁶−6.25×10⁴ VG/cells). SH-SY5Y KD cells were infected for 2 days then fixed and stained for SMN. The number of GEMS/cells was assessed by high-content imaging using the CX5 CellInsight.

FIG. 12 shows effect of empty particles on rAAVhu68-SMN1 potency. SH-SY5Y sh120 cells pre-treated with DOX (100 μg/mL) were plated at 1×10⁴ cells/well, infected with rAAVhu68-SMN1 (4×10⁶ VG/cell) and empty AAVhu68 particles at a final full:empty particles ratio of 1:1 and 1:3 per well. The mix of capsids was performed based on the AAV9 capsid titer.

FIG. 13A-B shows an exemplary image of Gemin 2-SMN1 staining in SH-SY5Y KD cells (knockdown with shRNA120) after 1:1 acetone:methanol fixation using the CX5 CellInsight. The merged images show an overlapping signal of SMN1 and Gemin 2.

FIG. 14 shows that SMN1 antibodies specifically detect bacterially purified SMN protein. 100 and 200 ng of purified SMN protein and 200 ng of an unrelated protein (RS1) were analyzed by SDS-PAGE and blotted with SMN1 antibodies. Mono.: monoclonal antibody; poly.: polyclonal antibody; I.B.: immunoblot.

FIG. 15 shows testing specificity and sensitivity of SMN1 antibodies by Western-Blot. 50 ng of bacterially purified SMN protein or 50 ng of HEK293T whole cell lysate were analyzed by SDS-PAGE and blotted with SMN1 antibodies described in the table above at 4° C. overnight. N.B.: Novus Biological; T.F.: Thermo-Fisher; mono.: monoclonal antibody; poly.: polyclonal antibody; I.B.: immunoblot.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About or approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Adeno-associated virus (AAV): As used herein, the terms “Adeno-associated virus” and “AAV” refer to viral particles, in whole or in part, of the family Parvoviridae and the genus Dependoparvovirus. AAV is a small replication-defective, nonenveloped virus. AAV includes, but is not limited to, AAV serotype 1, AAV serotype 2, AAV serotype 3 (including serotypes 3A and 3B), AAV serotypes 4, AAV serotypes 5, AAV serotypes 6, AAV serotypes 7, AAV serotypes 8, AAV serotypes 9, AAV serotypes 10, AAV serotypes 11, AAV serotypes 12, AAV serotype 13, snake AAV, avian AAV, bovine AAV, canine AAV, equine AAV, ovine AAV, goat AAV, shrimp AAV, and any variant of any of the foregoing. Wild-type AAV is replication deficient and typically requires co-infection of cells by a helper virus, e.g., adenovirus, herpes, or vaccinia virus, in order to replicate.

Aptamer: As used herein, the term “aptamer” refers to a macromolecule composed of nucleic acid (e.g., RNA, DNA) that binds tightly to a specific molecular target (e.g., an SMN polypeptide). A particular aptamer may be described by a linear nucleotide sequence and is typically about 15-60 nucleotides in length. Without wishing to be bound by any theory, it is contemplated that the chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers typically have very high affinities for their targets (e.g., affinities in the picomolar to low nanomolar range for proteins). In many embodiments, aptamers are chemically stable and can be boiled or frozen without loss of activity. Because they are synthetic molecules, aptamers are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Detection moiety: The term “detection moiety” as used herein refers to any element, molecule, functional group, compound, fragment or moiety that is detectable. In some embodiments, a detection moiety is provided or utilized alone. In some embodiments, a detection moiety is provided and/or utilized in association with (e.g., joined to) another agent (e.g., an antibody or an antigen-binding fragment thereof). Examples of detection moieties include, but are not limited to: various fluorescent dyes (such as, for example, fluorophores (e.g., Alexa-Fluor 488, FluoProbes 488, or DyLight 488), fluorescein dyes, acridine dyes, SYBR dyes, rhodamine dyes, oxazine dyes, etc.), ligands, radionuclides (e.g., ³H, ¹⁴C, ¹⁸F, ¹⁹F, ³²p, ³⁵S, ¹³⁵I, ¹²⁵I, ¹²³I, ⁶⁴Cu, ¹⁸⁷Re, ¹¹¹Y, ^{99m}Tc, ¹⁷⁷Lu, ⁸⁹Zr etc.), chemiluminescent agents (such as, for example, acridinum esters, stabilized dioxetanes, and the like), electrochemiluminescent agents (such as, for example, Sulfo Tags), bioluminescent agents (such as, for example, luciferin), spectrally resolvable inorganic fluorescent semiconductors nanocrystals (e.g., quantum dots), metal nanoparticles (e.g., gold, silver, copper, or platinum), nanoclusters, paramagnetic metal ions, enzymes (e.g., horseradish peroxidase, or alkaline phosphatase), colorimetric labels (e.g., dyes or colloidal gold), biotin, dioxigenin, haptens, and proteins for which antisera or monoclonal antibodies are available.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Expression: As used herein, the terms “expression” or “encoding” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Gene: As used herein, the term “gene” refers to a DNA sequence that codes for a product (e.g., an RNA product and/or a polypeptide product). In some embodiments, a gene includes coding sequence (i.e., a sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory elements that, for example, may control or effect one or more aspects of gene expression (e.g., inducible expression, etc.).

Gene therapy: As used herein, the term “gene therapy” refers to insertion or deletion of specific genomic DNA sequences to treat or prevent a disorder or condition for which such therapy is sought. In some embodiments, the insertion or deletion of genomic DNA sequences occurs in specific cells (e.g., target cells). Target cells may be from a mammal and/or may be cells in a mammalian subject. Mammals include but are not limited to humans, dogs, cats, cows, sheep, pigs, llamas, etc. In some embodiments, heterologous DNA is transferred to target cells. The heterologous DNA may be introduced into the selected target cells in a manner such that the heterologous DNA is expressed and a therapeutic product encoded thereby is produced. Additionally or alternatively, the heterologous DNA may in some manner mediate expression of DNA that encodes the therapeutic product, or it may encode a product, such as a peptide or RNA that in some manner mediates, directly or indirectly, expression of a therapeutic product. Genetic therapy may also be used to deliver nucleic acid encoding a gene product that replaces a defective gene or supplements a gene product produced by the mammal or the cell in which it is introduced. The heterologous DNA encoding the therapeutic product may be modified prior to introduction into the cells of the afflicted host in order to enhance or otherwise alter the product or expression thereof. Genetic therapy may also involve delivery of an inhibitor or repressor or other modulator of gene expression. Gene therapy may include in vivo or ex vivo techniques. In some embodiments, viral and non-viral based gene transfer methods can be used to introduce nucleic acids encoding a polypeptide of interest into mammalian cells or target tissues. Non-viral vector delivery systems include DNA plasmids, naked nucleic acid, and nucleic acid complexed with a delivery vehicle, such as poloxamers or liposomes. Viral vector delivery systems include DNA and RNA viruses, which have either episomal or integrated genomes after delivery to the cell. For a review of gene therapy procedures, see Anderson, Science 256:808-813 (1992); Miller, Nature 357:455-460 (1992); Feuerbach et al., Kidney International 49:1791-1794 (1996); Urnov et al., Nature Reviews Genetics 11, 636-646 (2010); and Collins et al., Proceedings Biologicial Sciences/The Royal Society, 282(1821):pii 20143003 (2015), each of which is hereby incorporated by reference in its entirety.

Helper Functions: As used herein, the term “helper functions” refer to functions that allow recombinant viral vectors (e.g., AAV) to be replicated and packaged by a host cell. Helper functions can be provided in any of a number of forms, including, but not limited to, as a helper virus or as helper virus genes, which aid in recombinant viral vector (e.g., AAV) replication and packaging. Helper virus genes include, but are not limited to, adenoviral helper genes such as E1A, E1B, E2A, E4 and VA. Helper viruses include, but are not limited to, adenoviruses, herpesviruses, poxviruses, such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

Host Cell: As used herein, the term “host cell” refers a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence).

“Improve,” “increase,” “inhibit,” or “decrease”: As used herein the terms “improve”, “increase,” “inhibit,” “decrease,” or grammatical equivalents thereof, indicate values that are relative to a baseline or other reference measurement. In some embodiments, an appropriate reference measurement may be or comprise a measurement in a particular system (e.g., in a single sample, e.g., of a culture medium) under otherwise comparable conditions absent presence of (e.g., prior to and/or after) a particular agent or treatment, or in presence of an appropriate comparable reference agent. In some embodiments, an appropriate reference measurement may be or comprise a measurement in a comparable system known or expected to respond in a particular way, in presence of the relevant agent or treatment.

Operably linked: As used herein, the term “operably linked” refers to a juxtaposition wherein the components described are in a relationship permitting them to function in their intended manner. In some embodiments, a regulatory element is “operably linked” to a functional element. In some such embodiments, an operably linked regulatory element is associated in such a way that expression and/or activity of the functional element is achieved under conditions compatible with the regulatory element. In some embodiments, “operably linked” regulatory elements are contiguous (e.g., covalently linked) with the coding elements of interest; in some embodiments, regulatory elements act in trans to or otherwise at a from the functional element of interest.

Payload: As used herein, the term “payload” refers to a nucleic acid sequence of interest (e.g., comprising a sequence that encodes a target payload, such as a target polypeptide) that is desired to be introduced into a cell, tissue, organ, organism, and/or system comprising cells. The payload can be a heterologous protein with a therapeutic purpose, e.g., an enzyme or antibody. The payload can be a heterologous nucleic acid with a therapeutic purpose, e.g., a CRISPR/Cas guide RNA. One of skill in the art will recognize that the payload can be selected from any heterologous protein or nucleic acid of interest.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (e.g., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: as used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Transduction: As used herein, the term “transduction” refers to the ability of a recombinant viral vector to enter one or more particular cell types and transfer the DNA contained within the recombinant viral vector into the cell. Transduction can be assessed by measuring the amount of recombinant viral DNA or RNA expressed from the recombinant viral DNA in a cell or population of cells, and/or by assessing the number of cells in a population that contain recombinant viral DNA or RNA expressed from the DNA. Transduction efficiency is a measure of the level of transduction from a starting amount of recombinant viral vector (e.g. the starting amount of vector being injected in vivo or applied to cells in vitro), and can be quantitative or qualitative, and/or with reference to a particular control, e.g. a prototypic recombinant viral vector. For example, if a candidate recombinant viral vector transduces twice as many cells as a control vector and/or the amount of recombinant viral DNA per cell from transduction with the candidate recombinant viral vector is twice that of transduction with the control vector, where the starting amount of each vector was the same (e.g., the amount of each vector injected into a subject or applied to cells was the same), it can be said that the transduction efficiency of the candidate recombinant viral vector is 200% greater than, or is twice that of, the transduction efficiency of the control vector.

Vector: As used herein, the term “vector” refers to a nucleic acid molecule capable of transporting another nucleic acid to which it has been linked. By way of non-limiting example, one type of vector is a viral vector, wherein additional DNA segments may be ligated into the viral genome. Another type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference in its entirety.

### DETAILED DESCRIPTION OF CERTAIN EMBODIMENTS

The present disclosure provides, inter alia, a quantitative cell-based in vitro assay for determining potency (e.g., biological activity) of a recombinant viral vector. The present disclosure is based, in part, on the discovery of an assay for determining potency, e.g., relative potency, of recombinant viral vectors (e.g., AAV vectors) encoding at least one payload (e.g., at least one SMN polypeptide) that is both quantitative and accurate. In particular, potency assays described herein are improved over prior methods that were long, cumbersome, and had relatively high failure rates. Thus, the present disclosure provides, inter alia, improved methods and compositions for determining potency of recombinant viral vectors that are useful in compositions for gene therapy and treating diseases and disorders (e.g., spinal muscular atrophy) with gene therapy methods.

Without wishing to be bound by theory, it is believed that, in some embodiments, (i) modified host cells with decreased expression of at least one payload (e.g., at least one SMN polypeptide) relative to an unmodified reference host cell of the same type; and/or (ii) detection of Gemini of coiled bodies (GEMs) presence, allows for improved potency assays of recombinant viral vectors described herein. In some embodiments, human modified host cells (e.g., a neuroblastoma cell line comprising a knockdown (e.g., constitutive or conditional) in a SMN gene (e.g., a SMN1 or SMN2 gene), e.g., SH-SY5Y KD cells) used in methods described herein are more physiologically relevant and/or easier to culture than other types of host cells (e.g., primary cells and/or non-human mammalian cells, e.g., mouse cells). In some embodiments, a SH-SY5Y KD cell comprises a knockdown (e.g., constitutive or conditional) in a SMN gene (e.g., a SMN1 or SMN2 gene), e.g., comprising or expressing an inhibitory nucleic acid against a SMN gene (e.g., a SMN1 or SMN2 gene), e.g., a shRNA against SMN1 (e.g., a doxycycline inducible shRNA against a SMN1 gene, e.g., shRNA120 or shRNA 128).

Recombinant Viral Vectors

The present disclosure, among other things, provides recombinant viral vectors (e.g., adeno-associated viral (AAV) vectors). Recombinant viral vectors have become widely used for inserting genes into mammalian cells (e.g., human cells). Many forms of vectors can be used to deliver a payload (e.g., at least one SMN polypeptide) described herein. Non-limiting examples of expression vectors include viral vectors (e.g., vectors suitable for gene therapy), plasmid vectors, bacteriophage vectors, cosmids, phagemids, and artificial chromosomes.

Non-limiting examples of viral vectors include, but are not limited to, adeno-associated virus (AAV), retrovirus (e.g., Moloney murine leukemia virus (MMLV), Harvey murine sarcoma virus, murine mammary tumor virus, or Rous sarcoma virus), adenovirus, SV40-type virus, polyomavirus, Epstein-Barr virus, papilloma virus, herpes virus, vaccinia virus, or polio virus.

In some embodiments, a recombinant viral vector comprises or is a retroviral vector. Retroviruses are enveloped viruses that belong to the viral family Retroviridae. Protocols for the production of replication-deficient retroviruses are known in the art (see, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991)). The recombinant virus can then be isolated and delivered to cells of the subject either in vivo or ex vivo. A number of retroviral systems are known in the art, for example See U.S. Pat. Nos. 5,994,136, 6,165,782, and 6,428,953. In some embodiments, the retrovirus comprises or is a lentivirus of the Retroviridae family. In some embodiments, the lentivirus comprises or is human immunodeficiency viruses (HIV-1 and HIV-2), simian immunodeficiency virus (S1V), feline immunodeficiency virus (FIV), equine infections anemia (EIA), or visna virus.

In some embodiments, a recombinant viral vector comprises or is an adenovirus vector. The adenovirus vector may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087, each of which is hereby incorporated by reference in its entirety. Further examples of adenoviral vectors can be found in U.S. Publication Nos. 20150093831, 20140248305, 20120283318, 20100008889, 20090175897 and 20090088398, each of which is hereby incorporated by reference in its entirety.

A recombinant viral vector may also be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819; the vectors and methods of their making are incorporated herein by reference in their entirety

In some embodiments, a recombinant viral vector is an AAV vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is hereby incorporated by reference in its entirety). Methods for generating and using AAV vectors are described, for example, in U.S. Pat. Nos. 5,139,941 and 4,797,368, each of which is hereby incorporated by reference in its entirety.

Generally, AAV vectors for use in the methods and compositions described herein may be of any AAV serotype. Several AAV serotypes have been characterized, including AAV1, AAV2, AAV3 (e.g., AAV3B), AAV4, AAV5, AAV6, AAV7, AAV8, AAV9, AAV10, and AAV11, as well as variants and/or hybrids thereof. For example, in some embodiments, an AAV vector is an AAV2/5, AAV2/6, AAV2/8 or AAV2/9 vector (e.g., AAV6, AAV8 or AAV9 serotype having AAV2 ITR). In some embodiments, an AAV9 variant includes those described in, e.g., WO 2016/049230, U.S. Pat. No. 8,927,514, US 2015/0344911, and U.S. Pat. No. 8,734,809, each of which is hereby incorporated by reference in its entirety.

In some embodiments, an AAV serotype may have or comprise a mutation in the AAV9 sequence as described by N Pulicherla et al. (Molecular Therapy 19(6): 1070-1078 (2011) which is hereby incorporated by reference in its entirety), such as but not limited to, AAV9.68, AAV9.9, AAV9.11, AAV9.13, AAV9.16, AAV9.24, AAV9.45, AAV9.47, AAV9.61, AAV9.84. In certain embodiments, an AAV9 variant comprises AAVhu68 or a variant thereof, as described in, e.g., WO 2018/160585, which is hereby incorporated by reference in its entirety. Other AAV vectors are described in, e.g., Sharma et al., Brain Res Bull. 2010 Feb. 15; 81(2-3): 273, which is hereby incorporated by reference in its entirety.

In some embodiments, an AAV vector comprises or is a naturally occurring AAV. In some embodiments, an AAV vector is a modified AAV (i.e., a variant of a naturally occurring AAV). In some embodiments, an AAV vector may be generated by directed evolution, e.g., by DNA shuffling, peptide insertion, or random mutagenesis, in order to introduce modifications into the AAV sequence to improve one or more properties for gene therapy, e.g., to avoid or lessen an immune response or recognition by neutralizing antibodies, and/or for more efficient and/or targeted transduction (Asuri et al., Molecular Therapy 20.2 (2012): 329-338). Methods of using directed evolution to engineer an AAV vector can be found, e.g., in U.S. Pat. No. 8,632,764. In some embodiments the modified AAV is modified to include a specific tropism.

AAV sequences of an AAV vector typically comprise the cis-acting 5′ and 3′ inverted terminal repeat sequences (See, e.g., B. J. Carter, in “Handbook of Parvoviruses”, ed., P. Tijsser, CRC Press, pp. 155-168 (1990), which is hereby incorporated by reference in its entirety). The ITR sequences are about 145 bp in length. In some embodiments, substantially the entire sequences encoding the ITRs are used in an AAV vector, although some degree of minor modification of these sequences can be permissible. Ability to modify these ITR sequences is known to those of skill in the art. (See, e.g., Sambrook et al, “Molecular Cloning. A Laboratory Manual”, 2d ed., Cold Spring Harbor Laboratory, New York (1989); and K. Fisher et al., J Virol, 70:520 532 (1996), which are each hereby incorporated by reference in their entirety). In some embodiments, an AAV vector of the present disclosure is a “cis-acting” plasmid containing a payload, in which the selected payload sequence and associated regulatory elements are flanked by the 5′ and 3′ AAV ITR sequences. The AAV ITR sequences may be obtained from any known AAV, including known mammalian AAV types and/or those described herein.

In some embodiments, an AAV vector may be a dual or triple AAV vector, e.g., for the delivery of large payloads (e.g., payloads of greater than approximately 5 kb) and/or to address safety concerns associated with administration of single AAV vectors. In some embodiments, a dual AAV vector may include two separate AAV vectors, each including a fragment of the full sequence of the large payload of interest, and when recombined, the fragments form the full sequence of the large payload of interest, or a functional portion thereof. In some embodiments, a triple AAV vector may include three separate AAV vectors, each including a fragment of the sequence of the large payload of interest, and when recombined, the fragments form the full sequence of the large payload of interest, or a functional portion thereof.

Multiple AAV (e.g., dual or triple AAV vectors) can be delivered to and co-transduced into the same cell, where the two or three fragments of payload recombine together and generate a single mRNA transcript of the entire large payload of interest. In some embodiments, fragmented payloads include a non-overlapping sequences. In some embodiments, fragmented payloads include a specified overlapping sequences. In some embodiments, multiple AAV vectors of dual or triple transfection may be the same type of AAV vector (e.g., same serotype and/or the same construct). In some embodiments, multiple AAV vectors of the dual or triple may be different types of AAV vector (e.g., different serotype or construct).

Exemplary AAV vectors useful in accordance with the present disclosure include single-stranded (ss) or self-complementary (sc) AAV nucleic acid vectors. In some embodiments, an AAV vector comprises a single-stranded (ss) or self-complementary (sc) AAV nucleic acid vector. In some embodiments, an AAV vector comprises an expression construct described herein and one or more regions comprising inverted terminal repeat (ITR) sequences (e.g., wild-type ITR sequences or engineered ITR sequences) flanking the expression construct. In some embodiments, an AAV vector is encapsidated by a viral capsid. In some embodiments, to viral capsid comprises 60 capsid protein subunits. In some embodiments, the viral capsid comprises VP1, VP2, and VP3. In some embodiments, VP1, VP2, and VP3 subunits are present in the capsid at a ratio of approximately 1:1:10, respectively.

In some embodiments, ITR sequences of an AAV vector of the present disclosure can be derived from any AAV serotype (e.g., AAV1, AAV2, AAV3 (e.g., AAV3B), AAV4, AAV5, AAV6, AAV7, AAV8, AAV9, AAV10, and AAV11, and variants and/or hybrids thereof) or can be derived from more than one serotype. In some embodiments, ITR sequences are derived from one or more other serotypes. ITR sequences and plasmids containing ITR sequences are known in the art and are commercially available (See, e.g., products and services available from Vector Biolabs, Philadelphia, Pa.; Cellbiolabs, San Diego, Calif.; Agilent Technologies, Santa Clara, Ca; and Addgene, Cambridge, Mass.; and described in Kessler et al. PNAS. 1996 Nov. 26; 93(24): 14082-7; Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus; and U.S. Pat. Nos. 5,139,941 and 5,962,313; each of which is hereby incorporated by reference in its entirety).

In some embodiments, an AAV vector may comprise or be based on a serotype selected from any of the following serotypes, and variants thereof, including, but not limited to: AAV9.68, AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVFS, AAV-h, AAVH-1/hu. 1, AAVH2, AAVH-5/hu.3, AAVH6, AAVhE1 0.1, AAVhER1.14, AAVhEr1 0.16, AAVhEr1 0.18, AAVhER1.23, AAVhEr1 0.35, AAVhEr1 0.36, AAVhEr1 0.5, AAVhEr1 0.7, AAVhEr1 0.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi. 1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In some embodiments, an AAV serotype may be AAVDJ or a variant thereof, such as AAVDJ8 (or AAV-DJ8), as described by Grimm et al. (Journal of Virology 82(12): 5887-5911 (2008)) or in U.S. Pat. No. 7,588,772, each of which are hereby incorporated by reference in their entirety. In some embodiments, an AAV serotype may comprise or have a sequence as described in, e.g., U.S. Pat. No. 6,156,303, which is hereby incorporated by reference in its entirety, or derivatives thereof. In some embodiments, an AAV serotype may be or comprise a sequence as described in International Application Publication No. WO2015121501, which is hereby incorporated by reference in its entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

In some embodiments, an AAV serotype may be from any number of species. For example, in some embodiments, an AAV may be an avian AAV (AAAV). In some embodiments, an AAV serotype may be or comprise a sequence as described in U.S. Pat. No. 9,238,800, which is hereby incorporated by reference in its entirety. In some embodiments, an AAV serotype may be a bovine AAV (BAAV). The BAAV serotype may be or comprise a sequence as described in U.S. Pat. No. 9,193,769, which is hereby incorporated by reference in its entirety. The BAAV serotype may be or have a sequence as described in U.S. Pat. No. 7,427,396, which is hereby incorporated by reference in its entirety. In some embodiments, an AAV may be a caprine AAV. The caprine AAV serotype may be or comprise a sequence as described in U.S. Pat. No. 7,427,396, which is hereby incorporated by reference in its entirety. The AAV serotype may also be a variant or hybrid of any of the foregoing.

In some embodiments, an AAV may be a serotype generated by the AAV9 capsid library with mutations in amino acids 390-627 (VP1 numbering) as described by Pulicherla et al. (Molecular Therapy 19(6): 1070-1078 (2011), which is hereby incorporated by reference in its entirety. In some embodiments, a serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (CI 531 A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A,G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, an AAV vector comprises a capsid that includes modified capsid proteins (e.g., capsid proteins comprising a modified VP3 region). Methods of producing modified capsid proteins are known in the art (See, e.g., US20130310443, which is hereby incorporated by reference in its entirety). In some embodiments, the AAV vector comprises a modified capsid protein comprising at least one non-native amino acid substitution at a position that corresponds to a surface-exposed amino acid (e.g., a surface exposed Tyrosine) in a wild-type capsid protein. In some embodiments, the AAV vector comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in the wild-type capsid protein, a non-lysine amino acid (e.g., a glutamic acid) at a position that corresponds to a surface-exposed lysine amino acid in the wild-type capsid protein, a non-serine amino acid (e.g., a valine) at a position that corresponds to a surface-exposed serine amino acid in the wild-type capsid protein, or a combination thereof. In some embodiments, an AAV vector comprises a capsid that includes modified capsid proteins having at least 1, 2, 3, 4, 5, 6, 7, 8, 9, 10, or more amino acid substitutions.

In some embodiments, an AAV vector comprises one or more regions comprising a sequence that facilitates expression of coding sequence of a gene of interest, e.g., expression control sequences operably linked to the coding sequence. Non-limiting examples of expression control sequences include promoters, insulators, silencers, response elements, introns, enhancers, initiation sites, termination signals, and poly(A) tails. Any combination of such control sequences is contemplated herein (e.g., a promoter and/or an enhancer). In some embodiments, the expression construct includes other regulatory elements, such as WPRE.

An AAV vector can include conventional control elements operably linked to a nucleic acid encoding any polypeptide or payload described herein, in a manner that permits transcription, translation and/or expression in a cell transfected with a vector described herein. Expression control sequences include appropriate transcription initiation, termination, promoter and enhancer sequences; efficient RNA processing signals, such as splicing and polyadenylation (polyA) signals (e.g., a rabbit β-globin polyA signal); sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters that are native, constitutive, inducible, and/or tissue-specific, are known in the art and may be included in a vector described herein. Examples of constitutive promoters include, but are not limited to, a retroviral Rous sarcoma virus (RSV) LTR promoter (optionally with the RSV enhancer), a cytomegalovirus (CMV) promoter (optionally with CMV enhancer), an SV40 promoter, and an dihydrofolate reductase promoter.

Inducible promoters allow regulation of gene expression and can be regulated by exogenously supplied compounds, environmental factors, such as temperature, or the presence of a specific physiological state (e.g., acute phase, a particular differentiation state of the cell, or in replicating cells only). Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system, an ecdysone insect promoter, a tetracycline-repressible system, a tetracycline-inducible system, a RU486-inducible system, and an rapamycin-inducible system. Still other types of inducible promoters that may be useful are regulated by a specific physiological state, such as temperature, acute phase, a particular differentiation state of the cell, or in replicating cells only.

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken β-actin promoter (CB7), a Cbh promoter, a pol II promoter, or a pol III promoter.

In some embodiments, a promoter is a tissue or cell-specific promoter. For example, neuronal-specific promoters may include, but are not limited to, human synapsin I (SYN) promoter (e.g., as described in Li et al., Proc Natl Acad Sci USA 1993; 90: 1460-1464, which is hereby incorporated by reference in its entirety), mouse calcium/calmodulin-dependent protein kinase II (CaMKII) promoter (e.g., as described in Mayford et al., Proc Natl Acad Sci USA 1996; 93: 13250-13255, which is hereby incorporated by reference in its entirety), rat tubulin alpha I (Tal) promoter (e.g., as described in Gloster et al., J Neurosci 1994; 14: 7319-7330, which is hereby incorporated by reference in its entirety), rat neuron-specific enolase (NSE) promoter (e.g., as described in Forss-Petter et al, Neuron 1990; 5: 187-197, which is hereby incorporated by reference in its entirety), and human platelet-derived growth factor-beta chain (PDGF) promoter (e.g., as described in Sasahara et al, Cell 1991; 64: 217-227, which is hereby incorporated by reference in its entirety).

In another embodiment, a native promoter or fragment thereof for a nucleic acid encoding any payload described herein may be used. In some embodiments, other native expression control elements, such as enhancer elements, polyadenylation sites, or Kozak consensus sequences, may also be used to mimic native expression.

In some embodiments, a payload in an AAV vector described herein can be of any length, e.g., between 2 and 10,000 nucleotides in length or any integer value there between. In some embodiments, a nucleic acid sequence encoding a payload comprises at least 20 nucleotides, at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 880 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 2000 nucleotides, at least 2500 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides. In some embodiments, a nucleic acid sequence encoding a payload comprises between 50 and 25,000 nucleotides in length, between 100 and 20,000 nucleotides in length, between 500 and 10,000 nucleotides in length, between 1,000 and 8,000 nucleotides in length, and/or between 2,000 and 5,000 nucleotides in length.

In some embodiments, the methods comprise optimizing a multiplicity of infection (MOI) of recombinant viral vectors described herein for transduction. In some embodiments, an MOI is selected that produces a linear range of assay results. In some embodiments, assaying multiple MOIs is achieved by serial dilution. In some embodiments, a host cell (e.g., a modified host cell comprising or having reduced expression of at least one SMN polypeptide, e.g. a SH-SY5Y KD cell as described herein) is transduced with a recombinant viral vector at different MOIs (e.g., about 3, about 4, about 5, about 6, about 7, about 8, about 9, or about 10 MOIs) achieved by serial dilution. In some embodiments an about 1.2 fold, about 1.4 fold, about 1.6 fold, about 1.8 fold, about 2 fold, about 2.2 fold, about 2.4 fold, about 2.2. fold, about 2.4 fold, or about 3 serial dilution or a combination thereof is used. In some embodiments, about 5 different MOIs are tested by serial dilution.

In some embodiments, a viral vector is added to cell culture at an MOI of about 6.1×10⁵ VG/cell to about 4.0×10⁶ VG/cell, e.g., about 6.1×10⁵ VG/cell, about 6.2×10⁵ VG/cell, about 6.3×10⁵ VG/cell, about 6.4×10⁵ VG/cell, about 6.5×10⁵ VG/cell, about 7.0×10⁵ VG/cell, about 7.5×10⁵ VG/cell, about 8.0×10⁵ VG/cell, about 8.5×10⁵ VG/cell, about 9.0×10⁵ VG/cell, about 9.5×10⁵ VG/cell, about 9.8×10⁵ VG/cell, about 1.0×10⁶ VG/cell, about 1.5×10⁶ VG/cell, about 1.6×10⁶ VG/cell, about 2.0×10⁶ VG/cell, about 2.5×10⁶ VG/cell, about 3.0×10⁶ VG/cell, about 3.5×10⁶ VG/cell, or about 4.0×10⁶ VG/cell.

In some embodiments, a viral vector is added to cell culture at an MOI of about 6.1×10⁵ VG/cell+/−50%, 6.1×10⁵ VG/cell+/−40%, 6.1×10⁵ VG/cell+/−30%, 6.1×10⁵ VG/cell+/−20%, 6.1×10⁵ VG/cell+/−10%, 6.1×10⁵ VG/cell+/−5%, or 6.1×10⁵ VG/cell+/−1%.

In some embodiments, a viral vector is added to cell culture at an MOI of about 9.8×10⁵ VG/cell+/−50%, 9.8×10⁵ VG/cell+/−40%, 9.8×10⁵ VG/cell+/−30%, 9.8×10⁵ VG/cell+/−20%, 9.8×10⁵ VG/cell+/−10%, 9.8×10⁵ VG/cell+/−5%, or 9.8×10⁵ VG/cell+/−1%.

In some embodiments, a viral vector is added to cell culture at an MOI of about 1.6×10⁶ VG/cell+/−50%, 1.6×10⁶ VG/cell+/−40%, 1.6×10⁶ VG/cell+/−30%, 1.6×10⁶ VG/cell+/−20%, 1.6×10⁶ VG/cell+/−10%, 1.6×10⁶ VG/cell+/−5%, or 1.6×10⁶ VG/cell+/−1%.

In some embodiments, a viral vector is added to cell culture at an MOI of about 2.5×10⁶ VG/cell+/−50%, 2.5×10⁶ VG/cell+/−40%, 2.5×10⁶ VG/cell+/−30%, 2.5×10⁶ VG/cell+/−20%, 2.5×10⁶ VG/cell+/−10%, 2.5×10⁶ VG/cell+/−5%, or 2.5×10⁶ VG/cell+/−1%.

In some embodiments, a viral vector is added to cell culture at an MOI of about 4×10⁶ VG/cell+/−50%, 4×10⁶ VG/cell+/−40%, 4×10⁶ VG/cell+/−30%, 4×10⁶ VG/cell+/−20%, 4×10⁶ VG/cell+/−10%, 4×10⁶ VG/cell+/−5%, or 4×10⁶ VG/cell+/−1%.

In some embodiments, MOIs are selected that produce a linear range of potency assay results. In other embodiments, MOIs are selected that produce a non-linear range of potency assay results. In some embodiments, a signal to noise ratio of a potency assay is about 2.5 or greater than 2.5, e.g., a signal to noise ratio of about 3.0, about 3.5, about 4.0, about 4.5, about 5.0, about 5.5, about 6.0, about 6.5, about 7.0, about 7.5, about 8.0, about 8.5, about 9.0, about 9.5, about 10, or greater. In some embodiments, a signal to noise ratio of about 2.5 or greater allows for use of a non-linear range of potency assay results.

Production of AAV Vectors

Methods for obtaining AAV vectors are known in the art. Typically, methods involve culturing a host cell which contains a nucleic acid sequence encoding an AAV capsid protein or fragment thereof; a functional rep gene; an AAV vector composed of AAV inverted terminal repeats (ITRs) and a payload; and/or sufficient helper functions to permit packaging of the recombinant AAV vector into the AAV capsid proteins AAV rep and cap genes may be from any AAV serotype for which recombinant virus can be derived, and may be from a different AAV serotype than the AAV genome ITRs, including, but not limited to, any AAV serotype described herein. Production of pseudotyped AAV is disclosed in, for example, international patent application publication number WO 01/83692, which is hereby incorporated by reference in its entirety.

Components to be cultured in a host cell to package an AAV vector in an AAV capsid may be provided to the host cell in trans. Alternatively, any one or more of required components (e.g., AAV vector, rep sequences, cap sequences, and/or helper functions) may be provided by a stable host cell that has been engineered to contain one or more of the required components using methods known to those of skill in the art. In some embodiments, such a stable host cell contains the required component(s) under the control of an inducible promoter. In other embodiments, the required component(s) may be under the control of a constitutive promoter. In other embodiments, a selected stable host cell may contain selected component(s) under the control of a constitutive promoter and other selected component(s) under the control of one or more inducible promoters. For example, a stable host cell may be generated comprising E1 helper functions under the control of a constitutive promoter and rep and/or cap proteins under the control of inducible promoters. Other stable host cells may be generated by one of skill in the art using routine methods.

Recombinant AAV, rep sequences, cap sequences, and helper functions required for producing an AAV vector of the disclosure may be delivered to a packaging host cell using any appropriate genetic element (e.g., vector). A selected genetic element may be delivered by any suitable method known in the art, e.g., to those with skill in nucleic acid manipulation and include genetic engineering, recombinant engineering, and synthetic techniques (See, e.g., Sambrook et al, Molecular Cloning: A Laboratory Manual, Cold Spring Harbor Press, Cold Spring Harbor, N.Y., which is hereby incorporated by reference in its entirety). Similarly, methods of generating AAV virions are well known and any suitable method can be used with the present disclosure (see, e.g., K. Fisher et al, J. Virol., 70:520-532 (1993) and U.S. Pat. No. 5,478,745, which are each hereby incorporated by reference in their entirety).

In some embodiments, AAV vectors may be produced using a triple transfection method (e.g., as described in U.S. Pat. No. 6,001,650, which is hereby incorporated by reference in its entirety). In some embodiments, AAV vectors are produced by transfecting a host cell with an AAV vector (comprising a payload) to be packaged into AAV particles, an AAV helper function vector, and an accessory function vector. An AAV helper function vector encodes helper function sequences (e.g., rep and cap), which function in trans for AAV replication and encapsidation. In some embodiments, the AAV helper function vector supports efficient AAV vector production without generating any detectable wild-type AAV virions (e.g., AAV virions containing functional rep and cap genes). Non-limiting examples of vectors suitable for use with the present disclosure include pHLP19 (see, e.g., U.S. Pat. No. 6,001,650, which is hereby incorporated by reference in its entirety) and pRep6cap6 vector (see, e.g., U.S. Pat. No. 6,156,303, which is hereby incorporated by reference in its entirety).

An accessory function vector encodes nucleotide sequences for non-AAV derived viral and/or cellular functions upon which AAV is dependent for replication (i.e., “accessory functions”). Accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

In some embodiments, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art (See, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197). Such techniques can be used to introduce one or more exogenous nucleic acids, such as a nucleotide integration vector and other nucleic acid molecules, into suitable host cells.

In some embodiments, a host cell is a mammalian cell. A host cell may be used as a recipient of an AAV helper construct, an AAV minigene plasmid, an accessory function vector, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which are hereby incorporated by reference in their entirety.

In some embodiments, a producer cell line is transiently transfected with a construct that encodes the payload flanked by ITRs and a construct(s) that encodes rep and cap. In another system, a packaging cell line that stably supplies rep and cap is transiently transfected with a construct encoding the payload flanked by ITRs. In each of these systems, AAV virions are produced in response to infection with helper adenovirus or herpesvirus, and rAAVs are separated from contaminating virus. Other systems do not require infection with helper virus to recover the AAV. In some embodiments, helper functions (e.g., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some embodiments, a payload flanked by ITRs and rep/cap genes are introduced into insect host cells by infection with baculovirus-based vectors. Such production systems are known in the art (see, e.g., Zhang et al., 2009, Human Gene Therapy 20:922-929, which is hereby incorporated by reference in its entirety). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which are hereby incorporated by reference in their entirety.

The foregoing methods for producing recombinant vectors are not meant to be limiting, and other suitable methods will be apparent to the skilled artisan.

Payload

An exemplary payload of interest is SMN (e.g., human SMN). Human SMN is a 38 kDa multifunctional protein ubiquitously expressed and found in both cytoplasm and nucleus, which is concentrated in the nucleus in distinct structures called Gemini of coiled bodies (GEMs) that reflect SMN mechanism of action. Particularly high levels of SMN expression are found in neuronal cells of the central nervous system (CNS). In cytoplasm, SMN plays a critical role in spliceosome assembly by interacting with proteins called Gemins (Gemins 2-8) to form the SMN complex. Once formed, the SMN complex is crucial by bringing together the Sm proteins and the small nuclear RNA (snRNA) to form small nuclear ribonucleoprotein (snRNP) indispensable to pre-mRNA processing to mRNA in the nucleus.

In some embodiments, a payload for production of a recombination viral rector comprises one or more nucleic acids encoding at least one SMN polypeptide. In some embodiments, at least one SMN polypeptide comprises or is a human SMN polypeptide.

In some embodiments, a payload comprises an SMN gene (e.g., a SMN1 or SMN2 gene) or a fragment thereof. In some embodiments, an SMN gene (e.g., a SMN1 or SMN2 gene) comprises a human SMN1 or SMN2 gene. In some embodiments, an SMN gene (e.g., a SMN1 or SMN2 gene) is codon optimized. In some embodiments, a SMN1 gene (e.g., a codon optimized SMN1 gene) comprises a nucleic acid sequence as disclosed in WO 2018/160585 (e.g., SEQ ID NO: 1 of WO 2018/160585). Exemplary human nucleic acid (GenBank Accession No. NM_000344.4), human amino acid sequence, and human codon optimized nucleic sequences of SMN1 are shown in Table 1.

TABLE 1
Human SMN-1 nucleic acid and amino acid sequences.

| | |
|---|---|
| Human SMN-1 | GCACCCGCGGGTTTGCTATGGCGATGAGCAGCGGCGGCA |
| nucleic acid sequence | GTGGTGGCGGCGTCCCGGAGCAGGAGGATTCCGTGCTGTT |
| (SEQ ID NO: 1) | CCGGCGCGGCACAGGCCAGAGCGATGATTCTGACATTTG |
| | GGATGATACAGCACTGATAAAAGCATATGATAAAGCTGT |
| | GGCTTCATTTAAGCATGCTCTAAAGAATGGTGACATTTGT |
| | GAAACTTCGGGTAAACCAAAAACCACACCTAAAAGAAAA |
| | CCTGCTAAGAAGAATAAAAGCCAAAAGAAGAATACTGCA |
| | GCTTCCTTACAACAGTGGAAAGTTGGGGACAAATGTTCTG |
| | CCATTTGGTCAGAAGACGGTTGCATTTACCCAGCTACCAT |
| | TGCTTCAATTGATTTTAAGAGAGAAACCTGTGTTGTGGTT |
| | TACACTGGATATGGAAATAGAGAGGAGCAAAATCTGTCC |
| | GATCTACTTTCCCCAATCTGTGAAGTAGCTAATAATATAG |
| | AACAAAATGCTCAAGAGAATGAAAATGAAAGCCAAGTTT |
| | CAACAGATGAAAGTGAGAACTCCAGGTCTCCTGGAAATA |
| | AATCAGATAACATCAAGCCCAAATCTGCTCCATGGAACTC |
| | TTTTCTCCCTCCACCACCCCCCATGCCAGGGCCAAGACTG |
| | GGACCAGGAAAGCCAGGTCTAAAATTCAATGGCCCACCA |
| | CCGCCACCGCCACCACCACCACCCCACTTACTATCATGCT |
| | GGCTGCCTCCATTTCCTTCTGGACCACCAATAATTCCCCC |
| | ACCACCTCCCATATGTCCAGATTCTCTTGATGATGCTGAT |
| | GCTTTGGGAAGTATGTTAATTTCATGGTACATGAGTGGCT |
| | ATCATACTGGCTATTATATGGGTTTCAGACAAAATCAAAA |
| | AGAAGGAAGGTGCTCACATTCCTTAAATTAAGGAGAAAT |
| | GCTGGCATAGAGCAGCACTAAATGACACCACTAAAGAAA |
| | CGATCAGACAGATCTGGAATGTGAAGCGTTATAGAAGAT |
| | AACTGGCCTCATTTCTTCAAAATATCAAGTGTTGGGAAAG |
| | AAAAAAGGAAGTGGAATGGGTAACTCTTCTTGATTAAAA |
| | GTTATGTAATAACCAAATGCAATGTGAAATATTTTACTGG |
| | ACTCTATTTTGAAAAACCATCTGTAAAAGACTGGGGTGGG |
| | GGTGGGAGGCCAGCACGGTGGTGAGGCAGTTGAGAAAAT |
| | TTGAATGTGGATTAGATTTTGAATGATATTGGATAATTAT |
| | TGGTAATTTTATGAGCTGTGAGAAGGGTGTTGTAGTTTAT |
| | AAAAGACTGTCTTAATTTGCATACTTAAGCATTTAGGAAT |
| | GAAGTGTTAGAGTGTCTTAAAATGTTTCAAATGGTTTAAC |
| | AAAATGTATGTGAGGCGTATGTGGCAAAATGTTACAGAA |
| | TCTAACTGGTGGACATGGCTGTTCATTGTACTGTTTTTTTC |
| | TATCTTCTATATGTTTAAAAGTATATAATAAAAATATTTA |
| | ATTTTTTTTTAAATTA |
| Human SMN-1 | MAMSSGGSGGGVPEQEDSVLFRRGTGQSDDSDIWDDTALIK |
| amino acid sequence | AYDKAVASFKHALKNGDICETSGKPKTTPKRKPAKKNKSQK |
| (SEQ ID NO: 2) | KNTAASLQQWKVGDKCSAIWSEDGCIYPATIASIDFKRETCV |
| | VVYTGYGNREEQNLSDLLSPICEVANNIEQNAQENENESQVS |
| | TDESENSRSPGNKSDNIKPKSAPWNSFLPPPPPMPGPRLGPGK |
| | PGLKFNGPPPPPPPPPPHLLSCWLPPFPSGPPIIPPPPPICPDSLD |
| | DADALGSMLISWYMSGYHTGYYMGFRQNQKEGRCSHSLN |
| Codon optimized | GGCCATGTCGAGTGGGGGCAGTGGAGGGGGAGTGCCAGA |
| human SMN-1 | ACAGGAAGATTCCGTGCTGTTCAGGCGAGGAACCGGGCA |
| nucleic acid sequence | GAGTGACGACAGTGACATTTGGGACGACACGGCCCTGAT |
| (SEQ ID NO: 3) | CAAGGCCTATGACAAAGCCGTGGCCTCCTTCAAGCACGC |
| | GCTGAAGAACGGCGACATTTGCGAAACCAGCGGCAAGCC |
| | TAAGACCACCCCTAAACGGAAGCCCGCCAAGAAAAATAA |
| | GTCCCAGAAAAAGAACACAGCCGCAAGTCTTCAGCAATG |
| | GAAGGTGGGGGATAAGTGCTCCGCGATATGGAGTGAAGA |
| | CGGGTGCATCTATCCTGCCACCATCGCCAGCATAGACTTC |
| | AAGCGCGAAACCTGCGTGGTGGTGTACACTGGATACGGG |
| | AACCGGGAGGAGCAGAACCTGAGCGACCTGTTGAGCCCT |
| | ATTTGTGAGGTGGCCAACAACATCGAGCAGAATGCGCAA |
| | GAAAATGAAAACGAGAGTCAGGTGTCCACCGATGAGAGT |
| | GAAAACAGTAGGAGCCCCGGCAACAAATCCGACAATATC |
| | AAGCCCAAAAGCGCACCCTGGAATAGCTTCCTTCCACCCC |
| | CCCCCCCAATGCCCGGACCTCGACTGGGCCCCGGAAAGC |
| | CTGGCCTGAAGTTCAACGGCCCCCCTCCTCCTCCTCCCCCT |
| | CCTCCCCCCCACCTGCTGAGCTGCTGGTTGCCCCCTTTCCC |
| | TTCGGGACCCCCTATCATACCTCCCCCCCCCCCTATTTGCC |
| | CTGACTCCCTGGACGACGCGGACGCGCTGGGCAGTATGCT |
| | CATCTCGTGGTACATGTCAGGATACCACACCGGGTACTAC |
| | ATGGGCTTCAGACAAAATCAGAAGGAAGGACGATGTAGT |
| | CACTCCCTGAAT |

Host Cells

The present disclosure, among other things, provides host cells (e.g., modified host cells comprising or having reduced expression of at least one SMN polypeptide, e.g. SH-SY5Y KD cells as described herein) for transduction with at least one viral vector described herein. A host cell includes a progeny cell of an original cell transfected with at least one vector described herein. A progeny cell of a parental cell may not be substantially identical in morphology or genomic content as a parent cell due to natural, accidental, or deliberate mutation.

The present disclosure recognizes that cells vary in permissivity to viral vectors. Further, the present disclosure recognizes that cells vary in their capability to transcribe and translate proteins encoded by viral vectors. Thus, without wishing to be bound by any particular theory, the present disclosure recognizes that host cells (e.g., modified host cells comprising or having reduced expression of at least one SMN polypeptide, e.g. SH-SY5Y KD cells as described herein) used for potency assays described herein must be highly permissive to viral vectors. Thus, in accordance with various embodiments, assays as described herein utilize a cell permissive for a viral vector encoding a payload of interest.

In some embodiments, a host cell comprises or is a mammalian cell. In some embodiments, a host cell comprises or is a human, monkey, ape, hamster, rat, or mouse cell. In some embodiments, a host cell comprises or is a human cell. In some embodiments, a host cell is not a non-human mammalian cell (e.g., a mouse cell). In some embodiments, a host cell comprises or is an immortalized cell. In some embodiments, a host cell comprises or is a tumor or myeloma cell. In some embodiments, a host cell comprises or is a cell line. In some embodiments, a host cell is not a primary cell. In some embodiments, a host cell is derived from neuronal tissue. In some embodiments, a host cell is derived from kidney tissue. In some embodiments, a host cell is derived from liver tissue. In some embodiments a host cell is derived from eye tissue.

In some embodiments, a host cell comprises or is a neuroblastoma cell (e.g., SH-SY5Y, B35, IMR-32 or SK-N-AS cell), a HeLa cell (e.g., HeLa-RC32 cell), a kidney cell (e.g., Huh7, HEK293, 293 EBNA, MSR 293, MDCK, HaK, Vero cell, CV1, or BHK cell), a CHO cell (e.g., a CHO K1, DXB-11 CHO, or Veggie-CHO cell), a COS cell (e.g., a COS-7 cell), a liver cell (e.g., a HepG2 cell), a retinal cell (e.g., a RPE1, R28, or MU-PH1 cell), a fibroblast cell (e.g., a NIH/3T3 cell), or a cell line or variant derived from an aforementioned cell. In some embodiments, a host cell comprises or is a neuroblastoma cell (e.g., a SH-SY5Y cell).

In some embodiments, a host cell comprises or is a modified host cell. In some embodiments, a modified host cell lacks or has reduced expression of a payload of interest (e.g., at least one SMN polypeptide). In some embodiments, a modified host cell comprises a host cell described herein (e.g., a mammalian, e.g., human host cell described herein; or e.g., a neuronal (e.g., neuroblastoma), e.g., mammalian neuronal (e.g., neuroblastoma), e.g., human neuronal (e.g., neuroblastoma) host cell described herein) that further comprises a knockdown (e.g., constitutive or conditional) in a SMN gene (e.g., a SMN1 or SMN2 gene). In some embodiments, the modified host cell comprises an inhibitory nucleic acid (e.g., shRNA) against a SMN gene (e.g., a SMN1 or SMN2 gene). In certain embodiments, a modified host cell comprises or is a SH-SY5Y cell (e.g., a SH-SY5Y KD cell). In some embodiments, a SH-SY5Y KD cell comprises a knockdown (e.g., constitutive or conditional) in a SMN gene (e.g., a SMN1 or SMN2 gene), e.g., comprising or expressing an inhibitory nucleic acid against a SMN gene (e.g., a SMN1 or SMN2 gene), e.g., a shRNA against SMN1 (e.g., a doxycycline inducible shRNA against SMN1, e.g., shRNA120 or shRNA 128), e.g., an shRNA as described in Jangi et al. Proc Natl Acad Sci.114(12):E2347-E2356, 2017, which is hereby incorporated by reference in its entirety. In some embodiments, SH-SY5Y cells are transduced with at least one lentiviral vector expressing a shRNA against a SMN1 gene (e.g., a doxycycline inducible shRNA against SMN1 gene, e.g., shRNA120 or shRNA 128), e.g., as described in Jangi et al. Proc Natl Acad Sci.114(12):E2347-E2356, 2017. In some embodiments, an inhibitory nucleic acid (e.g., shRNA) against SMN1 does not target or affect a recombinant viral vector described herein (e.g., an inhibitory nucleic acid, e.g., shRNA, does not target a payload (e.g., a codon optimized SMN1 gene) of a recombinant viral vector described herein). In some embodiments, a host cell or modified host cell described herein does not comprise (e.g., is not) a primary cell. In some embodiments, a host cell or modified host cell described herein comprises (e.g., is) a cell line, e.g., an immortalized/continuous cell line.

Modified host cells that lack or have reduced expression of a payload of interest (e.g., at least one SMN polypeptide), can be obtained by any suitable means, including a knock out or knock down (e.g., a conditional knock out or knock down) of a payload of interest (e.g., at least one SMN polypeptide). For example, a modified host cell can include a knock down of a payload of interest (e.g., at least one SMN polypeptide) using shRNA, siRNA, clustered regularly interspaced short palindromic repeats (CRISPR) transcription-activator like effector nuclease (TALEN), and/or zinc finger endonuclease (ZFN). In some embodiments, a modified host cell comprise an inhibitory nucleic acid against a payload of interest (e.g., at least one SMN polypeptide). In some embodiments, an inhibitory nucleic acid comprises a shRNA, siRNA, or a miRNA. In some embodiments, a host cell comprise at least one shRNA for knock down of a payload of interest (e.g., at least one SMN polypeptide). In some embodiments, a shRNA comprises shRNA120 or shRNA 128. In some embodiments, an inhibitory nucleic acid (e.g., shRNA) does not target a recombinant viral vector described herein. In some embodiments, an inhibitory nucleic acid (e.g., shRNA) does not target a payload (e.g., a codon optimized SMN1 gene) of a recombinant viral vector described herein.

In some embodiments, host cells (e.g., modified host cells comprising or having reduced expression of at least one SMN polypeptide, e.g. SH-SY5Y KD cells as described herein) are seeded and transduced within a relatively short time period, e.g., within about 1 hour, about 2 hours, about 3 hours, about 4 hours, about 5 hours, about 6 hours, about 7 hours, about 8 hours, about 9 hours, about 10 hours, about 11, hours, about 12 hours, about 13 hours, about 14 hours, about 15 hours, about 16 hours, about 17 hours, about 18 hours, about 19 hours, about 20 hours, about 21 hours, about 22 hours, about 23, or about 24 hour period.

In some embodiments, a modified host cell comprises a decreased level of expression of a payload of interest (e.g., at least one SMN polypeptide), e.g., a decreased level of expression of about a 10-fold, about a 15-fold, about a 20-fold, about a 25-fold, about a 30-fold, about a 40-fold, about a 50-fold, about a 60-fold, about a 65-fold, about a 70-fold, about an 80-fold, about a 85-fold, about a 90-fold, about a 95-fold, about a 96-fold, about a 97-fold, about a 98-fold, about a 99-fold, or more relative to an unmodified reference host cell of the same type.

In some embodiments, prior to transfection, host cells (e.g., modified host cells comprising or having reduced expression of at least one SMN polypeptide, e.g. SH-SY5Y KD cells as described herein) are passaged multiple times, e.g., between about 10 times and about 30 times. In some embodiments host cells are passaged at least 10 times, e.g., at least 10 times, at least 11 times, at least 12 times, at least 13 times, at least 14 times, at least 15 times, at least 16 times, at least 17 times, at least 18 times, at least 19 times, at least 20 times, at least 21 times, at least 22 times, at least 23 times, at least 24 times, at least 25 times, at least 26 times, at least 27 times, at least 28 times, at least 29 times, at least 30 times, or more. In some embodiments, prior to transfection, host cells are subjected to at least one freeze-thaw cycle, e.g., at least two, three, four, or more freeze-thaw cycles.

In some embodiments, host cells (e.g., modified host cells comprising or having reduced expression of at least one SMN polypeptide, e.g. SH-SY5Y KD cells as described herein) are seeded onto a substrate (e.g., a cell culture vessel) prior to transduction with a recombinant viral vector described herein. Host cells can be cultured in a cell culture vessel. Cell culture vessels can comprise a cell culture dish, plate, or flask. Exemplary cell culture vessels include 35 mm, 60 mm, 100 mm, or 150 mm dishes, multi-well plates (e.g., 6-well, 12-well, 24-well, 48-well, or 96 well plates), or flasks (e.g., T-flasks, e.g., T-25, T-75, or T-160 flasks), or shaker flasks. In some embodiments, cell culture vessels comprise or are glass-bottom assay plates.

In some embodiments, host cells (e.g., modified host cells comprising or having reduced expression of at least one SMN polypeptide, e.g. SH-SY5Y KD cells as described herein) are seeded at a certain density. In some embodiments, host cells are seeded at a density of about 5.0×10³ to about 5.0×10⁴ cells/well. In some embodiments, host cells are seeded at a density of about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, or about 5.0×10⁴ cells/well. In certain embodiments, host cells are seeded at a density of about 5.0×10³.

Determining Potency of Recombination Viral Vectors

In some embodiments, potency (e.g., biological activity) of a recombinant viral vector described herein is determined by a level of expression of a payload of interest (e.g., at least on SMN polypeptide). In some embodiments, a potency assay comprises: (i) transducing host cells (e.g., modified host cells comprising or having reduced expression of at least one SMN polypeptide, e.g. SH-SY5Y KD cells as described herein) with a recombinant viral vector described herein encoding a payload of interest (e.g., at least one SMN polypeptide); (ii) contacting host cells with a first agent (e.g., optionally comprising a detection moiety) for detection of a payload of interest; (iii) contacting host cells with a second agent (e.g., optionally comprising a detection moiety) for detection of a first agent; and (iv) detecting presence of Gemini of coiled bodies (GEMs). In some embodiments, host cells (e.g., human host cells, e.g., a neuroblastoma cell line, e.g., SH-SY5Y cells (e.g., SH-SY5Y KD cells) are modified to decrease expression of a payload of interest (e.g., at least one SMN polypeptide) relative to an unmodified reference host cell of the same type.

In some embodiments, an assay is used to determine potency of a polypeptide (e.g., at least one SMN polypeptide) encoded by a recombinant viral vector. In some embodiments, potency of a recombinant viral vector is determined by a fluorescent assay. In some embodiments, potency of a recombinant viral vector is determined by a colorimetric assay. In some embodiments, potency of a recombinant viral vector is determined by an enzymatic assay. In some embodiments, a potency assay comprises imaging (e.g., fluorescence imaging, e.g., High-Content Imaging (HCI)) for detection of GEMs. In some embodiments, potency as referred to herein refers to relative potency, e.g., relative to a reference standard.

Detection of SMN Polypeptide

In some embodiments, a payload of interest (e.g., at least one SMN polypeptide) is detected with a first agent in combination with a second agent. In some embodiments, a first agent is labeled. In some embodiments, a second agent is labeled.

In some embodiments, a first agent comprises an antibody or an antigen-binding fragment thereof that binds to a payload of interest (e.g., at least one SMN polypeptide). Exemplary anti-SMN antibodies or fragments thereof are known in the art and are commercially available (see, e.g., products available from Novus Biologicals, Littletown, Colo.; Thermo Fisher Scientific; Waltham, Mass.; and GenTex, Irvine, Calif.). In some embodiments, a first agent comprises an aptamer. In some embodiments, an aptamer comprises RNA, DNA, or a combination thereof. In some embodiments, an aptamer binds tightly to a payload of interest (e.g., at least one SMN polypeptide).

In some embodiments, a first agent comprises a detection moiety. In some embodiments, a first agent is covalently or non-covalently associated with a detection moiety. In some embodiments, a second agent comprises a detection moiety. In some embodiments, a second agent is covalently or non-covalently associated with a detection moiety. In some embodiments, a first agent and a second agent comprise a detection moiety. In some embodiments, a first agent and a second agent are covalently or non-covalently associated with a detection moiety.

A detection moiety may comprise any element, molecule, functional group, compound, fragment, or moiety that is detectable. In some embodiments, a detection moiety comprises or is a fluorescent dye (e.g., a fluorescein dye, acridine dye, SYBR dye, rhodamine dye, or oxazine dye), radionuclide (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, or ⁸⁹Zr) chemiluminescent agent (e.g., acridinum ester or stabilized dioxetanes), electrochemiluminescent agent (e.g., Sulfo Tags), bioluminescent agent (e.g., luciferin), inorganic fluorescent semiconductors nanocrystal (e.g., quantum dots), metal nanoparticle (e.g., gold, silver, copper, or platinum), nanocluster, paramagnetic metal ion, enzyme (e.g., horseradish peroxidase, alkaline phosphatase, etc.), colorimetric label (e.g., dye or colloidal gold), or a derivative of any of the foregoing. In some embodiments, a detection moiety comprises or is a fluorescent dye or a derivative thereof. In some embodiments, a detection moiety comprises or is a fluorophore (e.g., Alexa-Fluor 488, FluoProbes 488, or DyLight 488).

Detection of Gemini of Coiled Bodies (GEMs)

Human SMN polypeptide is ubiquitously expressed and found in both cell cytoplasm and nucleus. In the nucleus, SMN is concentrated in distinct structures called Gemini of coiled bodies (GEMs). In some embodiments, presence of GEMS is detected by visual inspection, e.g., as a result of the bright, dense GEMs structures inside nuclei.

In some embodiments, presence of GEMs is detected by imaging. In some embodiments, imaging comprises fluorescence imaging (e.g., High-Content Imaging (HCI)). For example, HCI describes a set of analytical methods using automated microscopy, multi-parameter image processing, and visualization tools to extract quantitative data from cell populations. HCI can be used to report quantitatively on parameters such as, but not limited to, spatial distribution of targets in individual cells or cells structures. In some embodiments, HCI comprises fluorescence imaging of samples in a high-throughput format. In some embodiments, presence of GEMs is detected using HCI. In some embodiments, HCI detects changes in a host cell (e.g., a modified host cell comprising or having reduced expression of at least one SMN polypeptide, e.g. a SH-SY5Y KD cell as described herein) at a subcellular level (e.g., presence of GEMs in nuclei).

In some embodiments, presence of GEMs is detected by assaying markers of GEMs (e.g., one or more of Gemins 2-8 polypeptides). In some embodiments, a marker of GEMs indicates co-localization with GEMs. In some embodiments, a marker of GEMs comprises a Gemin 1 polypeptide, Gemin 2 polypeptide, Gemin 3 polypeptide, Gemin 4 polypeptide, Gemin 5 polypeptide, Gemin 6 polypeptide, Gemin 7 polypeptide, and/or Gemin 8 polypeptide. In some embodiments, markers of GEMs are assayed by imaging. In some embodiments, markers of GEMs are assayed by fluorescence imaging (e.g., High-Content Imaging (HCI)).

In some embodiments, potency assays described herein indicate stability of a recombinant viral vector (e.g., comprising a payload encoding a SMN polypeptide). In some embodiments, potency is determined following thermal stress of a recombinant viral vector. In some embodiments, a recombinant viral vector is subjected to at a particular temperature (e.g., causing thermal stress) for a particular length of time. In some embodiments, a recombinant viral vector is subjected to a temperature of about 40° C. to about 80° C., e.g., about 40° C., about 45° C., about 45° C., about 50° C., about 55° C., about 60° C., about 65° C., about 70° C., about 75° C., or about 80° C.). In some embodiments, a recombinant viral vector is held at particular temperature (e.g., causing thermal stress) for 1 minutes, 2 minutes, 5 minutes, 10 minutes, 15 minutes, 20 minutes, 25 minutes, 30 minutes, 45 minutes, 60 minutes, or longer.

In some embodiments, potency of a recombinant viral vector (e.g., comprising a payload encoding a SMN polypeptide) does not decrease in the presence of empty capsids. In some embodiments, a recombinant viral vector comprises a plurality of empty virus capsids. In some embodiments, a recombinant viral vector comprises a plurality of empty virus capsids (e.g., empty AAV capsids), e.g., a ratio of empty capsids to capsids comprising recombinant viral vector is about 2:1 to about 1:10, e.g., about 1:1 to about 1:3. In some embodiments, a recombinant viral vector preparation (e.g., AAV preparation) comprises 30% or less empty capsids, e.g., 40%, 25%, 20%, 15%, 10%, 7.5%, 5%, 2.5%, 1%, or less empty capsids.

In some embodiments, relative potency of a recombinant viral vector is determined, e.g., by parallel line analysis (PLA) against a standard curve, e.g., of a reference standard after linear regression data fit. In some embodiments, relative potency of a recombinant viral vector is at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.9%, at least 100%, at least 110%, at least 120%, at least 130%, at least 140%, at least 150%, or higher relative to a reference standard. Any suitable reference standard may be used. As used herein, a “reference standard” refers to a composition comprising a recombinant viral vector (e.g., encoding a SMN polypeptide), whose concentration and/or potency is known.

In some embodiments, assays described herein determine potency with high accuracy. In some embodiments, potency is determined with an accuracy of about 90%, about 91%, about 92%, about 93%, about 94%, about 95%, about 96%, about 97%, about 98%, about 99%, or more. In some embodiments, potency is determined with a precision of about 5%, about 6%, about 7%, about 8%, about 9%, or about 10%.

Uses

The present disclosure, among other things, provides methods of delivering a gene therapy to a cell or tissue. In particular, the present disclosure provides methods of treating a subject with a composition (e.g., a pharmaceutical composition) comprising a plurality of recombinant viral vectors (e.g., AAV vectors) assayed for potency with methods described herein.

In some embodiments, methods and kits of the present invention may be used for the evaluation and/or monitoring of gene therapy. In some embodiments, gene therapy comprises administration of a composition (e.g., a pharmaceutical composition) comprising a plurality of recombinant viral vectors (e.g., AAV vectors) that have been assayed with the methods described herein. In some embodiments, samples for evaluating and/or monitoring gene therapy may be obtained prior to the initiation of gene therapy. In some embodiments, samples are obtained after a first gene therapy treatment or dose. In some embodiments, samples are obtained after the conclusion of gene therapy. In some embodiments, samples are obtained at specific time points, intervals, or any other metric of time before, during, or after gene therapy is performed.

In some embodiments, a composition (e.g., a pharmaceutical composition) comprising a plurality of recombinant viral vectors (e.g., AAV vectors) assayed for potency with methods described herein is administered to a subject suffering from or at risk of a disease, disorder, or condition. In some embodiments, a composition (e.g., a pharmaceutical composition) comprising a plurality of recombinant viral vectors (e.g., AAV vectors) described herein is administered in combination with one or more additional therapeutics agents to a subject. In some embodiments, a composition (e.g., a pharmaceutical composition) comprising a plurality of recombinant viral vectors (e.g., AAV vectors) described herein is contacted with an organ, tissue, or cells ex vivo. The organ, tissue, or cells can be introduced into a subject and can be protected from damage that would otherwise be caused by the recipient's immune system.

In some embodiments, a disease or disorder comprises or is a motor neuron disease or disorder, e.g., a disease or disorder that affects one or more functions of motor neurons. In some embodiments, a protein deficiency or dysfunction in Central Nervous System (CNS) motor neurons causes a motor neuron disease or disorder. In some embodiments, motor neurons are in brain tissue. In some embodiments, motor neurons are in spinal cord tissue. Exemplary motor neuron diseases and disorders include, but are limited to, Spinal Muscular Atrophy (SMA), Amyotrophic Lateral Sclerosis (ALS), Primary Lateral Sclerosis (PLS), Pseudobulbar Pasly, Hereditary Spastic Paraplegia, Progressive Muscular Atrophy (PMA), Progressive Bulbar Palsy (PBP), and Distal Hereditary Motor Neuropathy.

Brain regions contemplated for delivery of a composition (e.g., a pharmaceutical composition) comprising a plurality of recombinant viral vectors (e.g., AAV vectors) described herein include, but are not limited to, the motor cortex and/or the brain stem. In some embodiments, a composition (e.g., a pharmaceutical composition) comprising a plurality of recombinant viral vectors (e.g., AAV vectors) as described herein is delivered to the spinal cord. In some embodiments, a composition (e.g., a pharmaceutical composition) comprising a plurality of recombinant viral vectors (e.g., AAV vectors) as described herein is delivered to a lower motor neuron. In some embodiments, a composition (e.g., a pharmaceutical composition) comprising a plurality of recombinant viral vectors (e.g., AAV vectors) as described herein is delivered to a nerve cell, glial cell, and/or Schwann cell. In some embodiments, a glial cell comprises or is a microglial cell, an oligodendrocyte, or an astrocyte.

In some embodiments, a composition (e.g., a pharmaceutical composition) comprising a plurality of recombinant viral vectors (e.g., AAV vectors) as described herein is administered to a subject having or at risk of SMA. In some embodiments, a subject exhibits motor neuron degeneration in spinal cord and/or skeletal muscle atrophy. In some embodiments, a subject exhibits one or more symptoms comprising muscle weakness, physical disabilities, and/or an increased risk of death.

SMA is one of the most commonly inherited progressive genetic neuromuscular disease-causing death in childhood with an incidence of 1 in 10,000 in the US. In some embodiments, a subject is a child. In some embodiments, a subject is an adolescent. SMA has a broad range of age onset, severity, rate of progression, and variability between and within subtypes. Four subtypes, SMA type 1 to 4, are classified with type 1 being the most severe type associated with the worst prognosis and death within two years of age. In some embodiments, SMA comprises or is SMA type 1, SMA type 2, SMA type 3, SMA type 4, or a combination thereof. In some embodiments, a subject comprises a homozygous mutation of a SMN1 gene. In some embodiments, a subject has reduced expression of a SMN polypeptide, e.g., relative to a subject with a wildtype SMN1 gene.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. An example is provided for illustrative purposes only. It is not to be construed as limiting the scope or content of the disclosure in any way.

### EXAMPLE

The following example is put forth so as to provide those of ordinary skill in the art with a complete disclosure and description of how to make and use the present invention, and is not intended to limit the scope of what the inventors regard as their invention nor are they intended to represent that the experiments below are all or the only experiments performed.

### Example 1: Potency Assay Development

The present example demonstrates development of a robust potency assay. The example describes the critical reagents and processes to determine the potency of SMN protein encoded by an AAVhu68 viral vector.

1.1 SMN Protein Detection with Anti-SMN Antibody

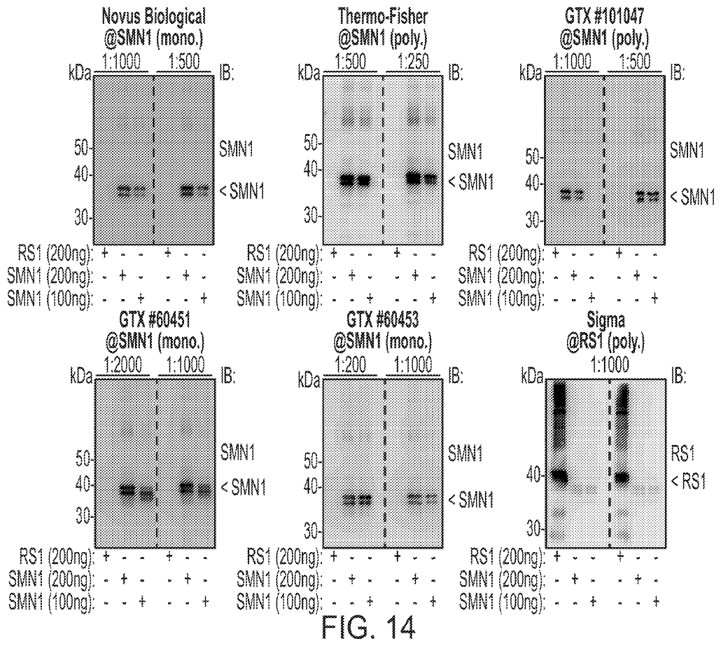

Antibody specificity is essential for the performance of High-Content Imaging (HCI) assay. The specificity of five different commercially available antibodies (Table 2) was tested by Western Blot against commercially available purified SMN protein produced in E. coli (Origene). As shown by Western Blot in FIG. 14, those five antibodies (tested at different dilutions based on manufacturer's recommendations) recognized purified SMN protein (100 and 200 ng) but not RS1 protein used as a negative control.

TABLE 2
SMN1 antibodies selected for SMN1 potency
assay development.

| Name in MDR | Company | Clone | Species |
|---|---|---|---|
| Novus Biological | Novus Biological | 2B1 | Mouse |
| Thermo-Fisher | Thermo-Fisher | — | Rabbit |
| GTX60451 | GeneTex | 2F1 | Mouse |
| GTX60453 | GeneTex | 5H1 | Mouse |
| GTX101047 | GeneTex | — | Rabbit |

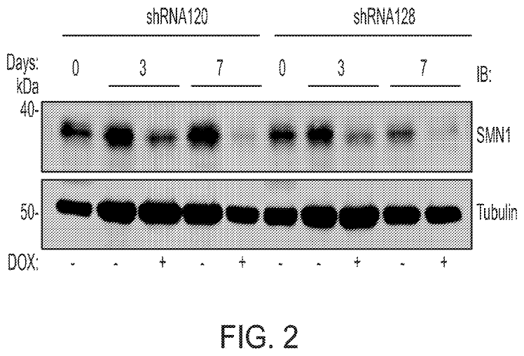

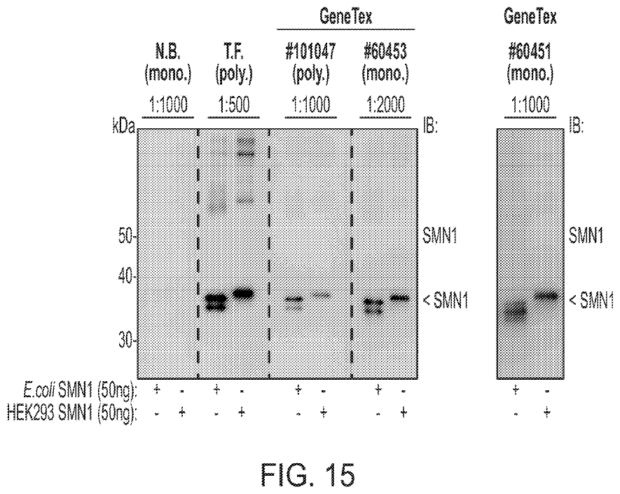

The specificity of the antibodies was further evaluated against HEK293 whole cell lysate (50 ng) expressing a high level of endogenous SMN protein. As shown in FIG. 15 antibodies purchased from GeneTex and Thermo-Fisher appear to detect both bacterially purified and endogenous SMN protein. Moreover, the sensitivity of the three GeneTex antibodies was higher compared to the monoclonal Novus Biological SMN1 antibody, which can't detect as low as 50 ng of purified SMN1 protein or endogenous SMN protein in HEK293 cell lysate (FIG. 2).

### 1.2 Cell Line Selection

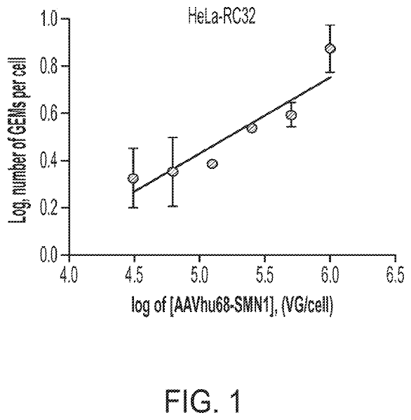

The next step was to determine which type of cells to use. The cell line needs to be easy to culture, easy to infect and if possible relevant to the mechanism of action of the disease of interest. Cell lines tested included HeLa-RC32 and SH-SY5Y. Using the HeLa-RC32 cell line was challenging due to a high endogenous expression of SMN, the background signal detected by HCI is high, increasing the variability of the results as shown in FIG. 1.

The human neuroblastoma cell line SH-SY5Y was previously used as a cell culture model representative of SMA to assess the effects of acute SMN loss in-vitro. The neuronal SH-SY5Y cell line is relevant to SMN mechanism of action and permissive to AAV9 vectors. SH-SY5Y cells conditionally knock-downed for SMN (SH-SY5Y KD cells, e.g., as described in Jangi et al. Proc Natl Acad Sci.114(12):E2347-E2356, 2017) showed a significantly reduced SMN background detected by HCI and in consequence the assay variability.

To confirm the efficiency of the conditional SMN knock-down, engineered SH-SY5Y cell line, SH-SY5Y shRNA120 and SH-SY5Y shRNA 128 cells (two SMN KD cell lines expressing two different shRNA sequences) were seeded and treated with DOX for 3 and 7 days. However, 7 days of DOX treatment will not be used for further development as the cells look unhealthy. The expression of SMN assessed by Western blotting (FIG. 2) shows a significant decrease of SMN endogenous protein after 3 days of DOX treatment in SH-SY5Y shRNA120 and SH-SY5Y shRNA 128 cells compare to day 0 (untreated cells). In conclusion, SMN expression is efficiently knocked-down by both shRNAs.

### 1.3 Optimization of Infection Parameters

Determining the Optimal Passage of SH-SY5Y Sh120 Cells

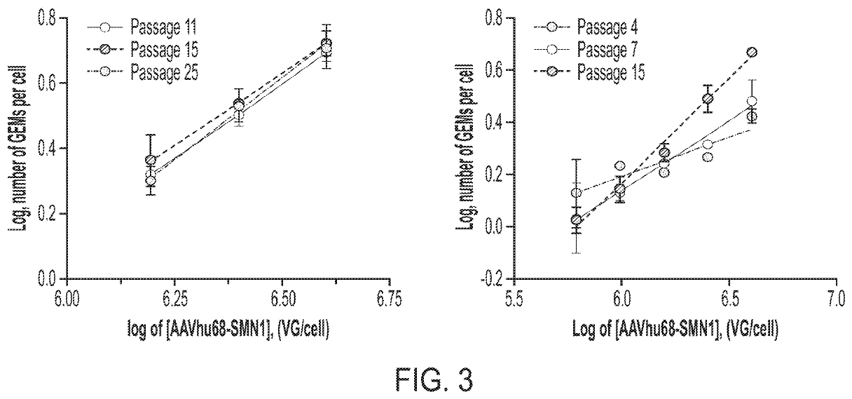

To investigate an effect of cell passage on GEM formation the engineered SH-SY5Y sh120 cells at different passages were treated with doxycycline for 3 days and frozen. The assay was performed as described in the detailed description of FIG. 3. The data obtained using cells from earlier passages had lower signal and slope compared to the cells at passage 11, 15, and 25 (higher passages were not tested). Therefore, only cells between passage 11 and 25 were used in this assay.

Plating SH-SY5Y Sh120 Cells In-Culture Vs. Cells from a Frozen State

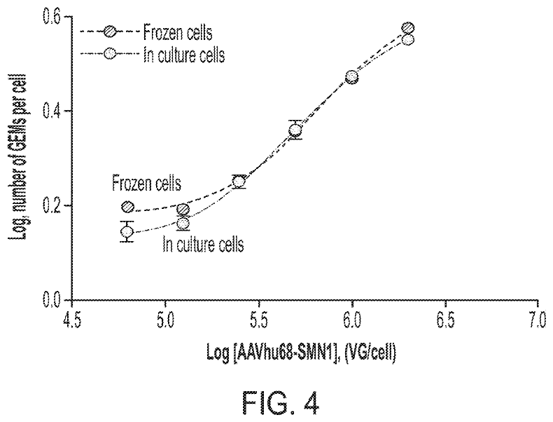

Cells are often the most variable and unpredictable component of a cell-based bioassay and cells in culture are prone to higher variability. The performance of the cells maintained in culture and cells thawed and plated from a frozen state (“thaw-and-go” cells) was compared. No significant differences were seen for SH-SY5Y sh120 cells (FIG. 4). While, the “thaw-and-go” cells require maintaining an ample cell bank and evaluation of each cell bank for the formation of GEMs after rAAVhu68-SMN1 infection, this method is more convenient for analysts, reduces assay time, synchronizes cells, and eliminates variability due to passaging. Therefore, “thaw-and-go” cells were used in the assay.

Optimization of Plating Density in 96-Well Plate

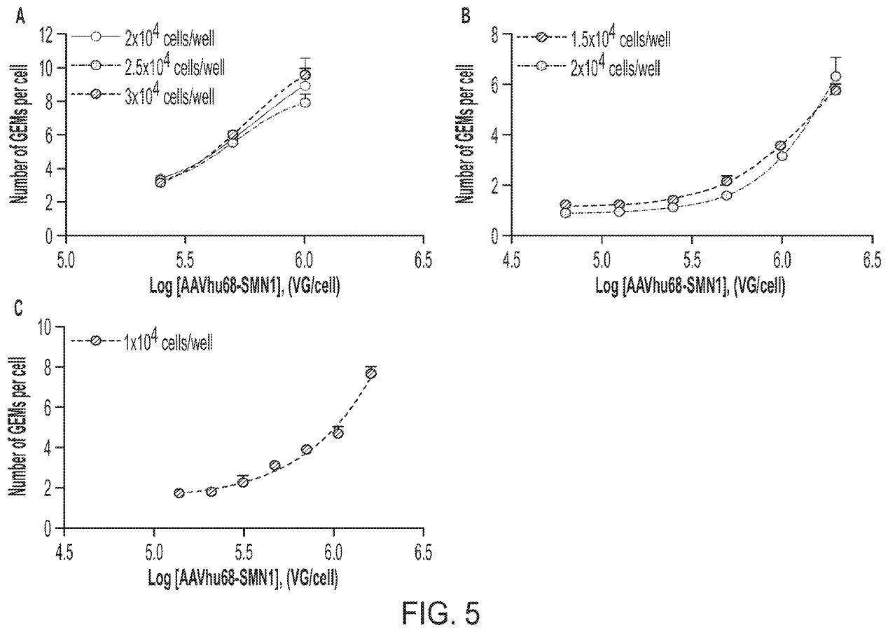

To determine the optimum cell density to use for the potency assay, the SH-SY5Y sh120 cells were plated at 6 different cell densities per well in a 96-well plate. The number of GEMs formed per cell at different rAAVhu68-SMN1 MOI was investigated. The preliminary experiments during method development performed with 2×10⁴ cells per well were used as the reference in those experiments. As shown in FIG. 5, for a given MOI, a good signal was achieved for all cell densities tested. Based on these results the lower cell density of 1×10⁴ cells/well was used for assay development in order to save vector material.

Optimization of Infection Time

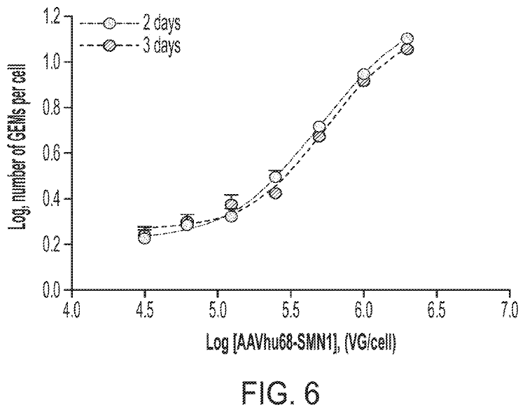

Infection of the SH-SY5Y sh120 cells with rAAVhu68-SMN1 was optimized to determine whether infecting cells for 3 days produces better curves or signal-to-background ratio. As shown below in FIG. 6, incubating cells for 3 days post-infection does not increase the number of GEMS formed per cells but extends the length of the assay. Infecting cells for 2 days was used for further development.

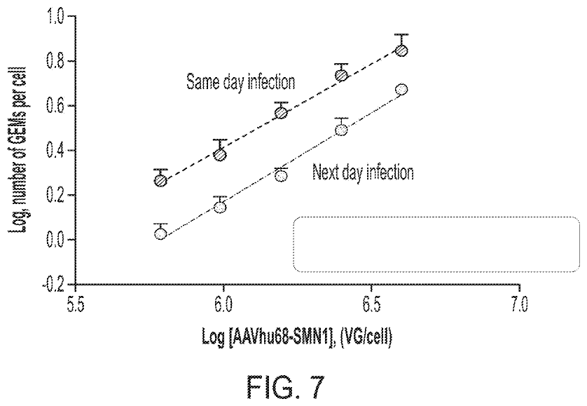

To further streamline the assay, the same-day versus next-day infection protocols were compared. The number of GEMs formed by cells plated and infected the same day was higher compared to cells infected the next day, while the dynamic range of the assay remained similar (FIG. 7). Moreover, plating and infecting cells the same day also decreases the length of the experiment and allows three assays per week instead of two. Therefore, same day infection was used for further development work.

Co-Infection of rAAVhu68-SMN1 with Adenovirus 5 not Needed

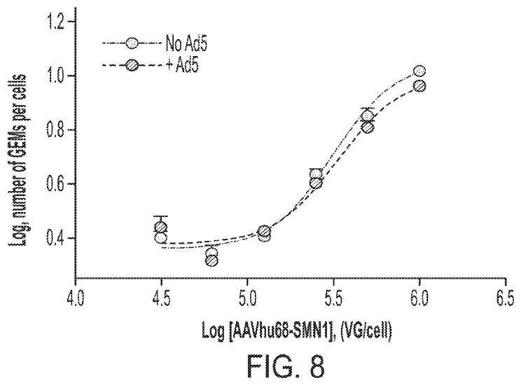

Human adenovirus 5 (Ad5) is used as a helper virus in the production of rAAVhu68-SMN1 by producer cell line. This type of approach not only produces rAAVhu68-SMN1 but also adenovirus particles requiring an extra purification step to eliminate the unwanted helper virus. However, some samples might contain residual Ad5 post-purification. To determine if Ad5 might affect the outcome of the potency assay, SH-SY5Y sh120 cells were co-infected in presence or absence of human adenovirus 5 (FIG. 8). No significant difference was seen in the presence Ad5 at 50 MOI.

### 1.4 Immunostaining Optimization

Optimization of the Primary and Secondary Antibodies

To improve GEMs staining and detection in SH-SY5Y KD cells, two concentrations of the primary antibody (1:250 and 1:500) were tested with two concentrations of three different secondary antibodies (Alexa-Fluor 488 goat anti-mouse, Alexa-Fluor 488 IgG1 goat anti-mouse and Alexa-Fluor 555 goat anti-mouse; 1:500 and 1:1,000). Following staining, the number of GEMs detected per cells was similar independently of the combination primary/secondary antibody used. In conclusion, the primary and secondary antibody were used at a 1:500 dilution during assay development and were used in combination to the same concentration for the assay.

Cell Fixation

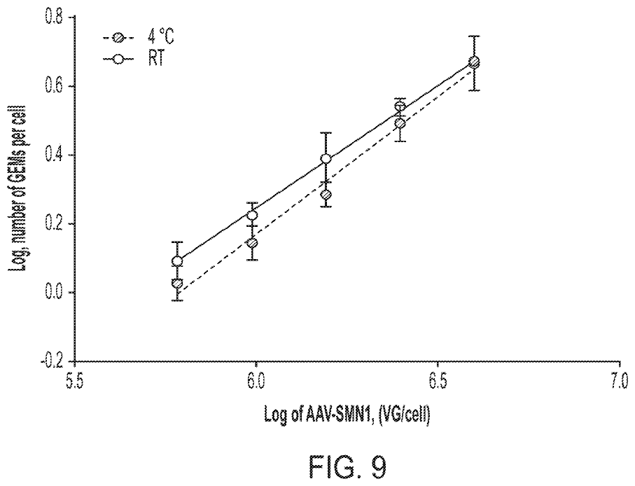

The quality of the cell fixation determines the quality of the immunofluorescence and consequently the outcome of the potency assay. The temperature of fixation is an important parameter which influence the efficiency of fixation, the quality of the pictures and can affect cell morphology (blebbing, vacuole formation etc.). The number of GEMs detected per cell for different MOI of rAAVhu68-SMN1 was comparable between cells fixed in 4% PFA/PBS at 4° C. or RT (FIG. 9). Consequently, cell were fixed at RT as it is more user-friendly.

Blocking Buffers

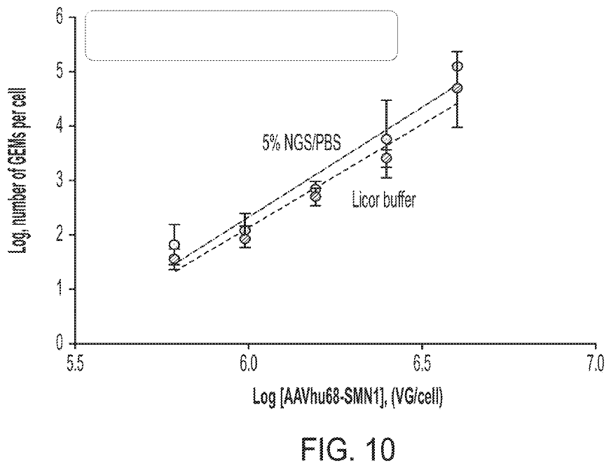

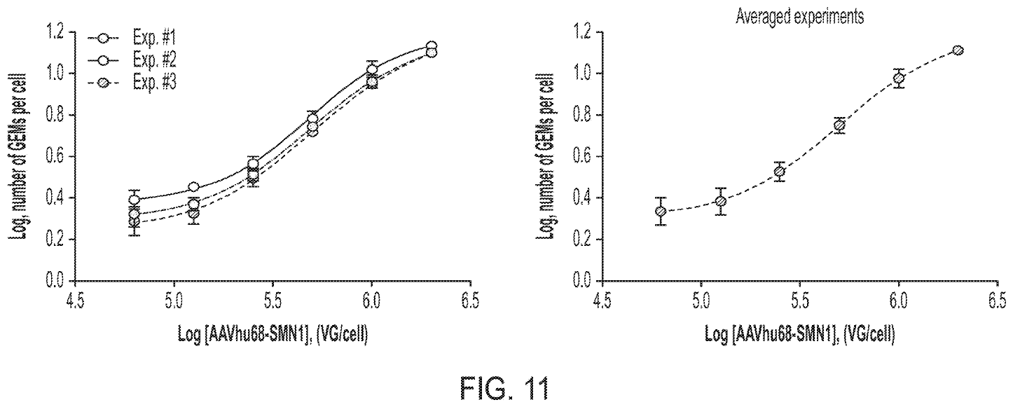

Blocking buffer is essential for preventing non-specific binding of antibodies, which improves the quality of immunostaining. If the blocking is partial or inadequate, the antibodies may bind a variety of sites that are not related to specific-antibody-antigen reactivity. Blocking with LI-COR buffer was compared to house-made 5% Normal Goat Serum (NGS)/PBS. The number of GEMs detected per cell for different MOI of rAAVhu68-SMN1 was comparable between cells incubating in LI-COR blocking reagent or 5% NGS/PBS (FIG. 10). In consequence, cell were incubated in 5% NGS/PBS blocking reagent as the composition is fully characterized and controlled.

### 1.5 Data Acquisition and Analysis

The neuroblastoma SH-SY5Y KD cell line was plated at a relatively lower cell density of 1×10⁴ cells/well. Consequently, the number of pictures acquired was increased from 20 to 30 fields/well for more representative analysis.

Parameters to detect and quantify the GEMs are essential to the quality and reliability of the assay. Two main parameters of the analysis are the area of quantification and the method of GEMs quantification. As the formation of the SMN complex takes place in cell cytoplasm, the area of GEMs quantification included the nucleus and 8 pixels outside of the nucleus. Restricting the analysis to 4 pixels outside of the nucleus or the nucleus only proportionally decreases the number of GEMs/cell but does not affect the outcome of the assay.

The “Local Maxima” method is a peak detection method to determine the number of GEMs which identifies spikes in pixel intensity within the spot detection region. Based on this method, many peaks (one peak=one object) can be detected within one spot area, increasing the number of objects detected per cells. A “Box” method that identifies spot as an area that can be counted and measured for size, shape, and intensity was implemented. By using this method, the number of objects detected per cell decreases but was considered to be more representative of the number of GEMs/cell. Moreover, as expected no GEMs were detected in DOX treated cells. In conclusion, the GEMs were quantified in the nucleus plus 8 pixels outside of it by using a “Box” method analysis.

Many biological responses fit into sigmoidal curve. A large range of rAAVhu68-SMN1 MOI (6.25×10⁴ to 8×10⁶ VG/cell) was tested to reach the upper limit of the number of GEMs formed per cell. However, the upper asymptote of the curve was not consistently reached. Consequently, the linear portion of the curve (6×10⁵ to 4×10⁶ VG/cell) was used for Parallel Line Analysis.

### 1.6 Reproducibility of the Potency Assay

To assess reproducibility the assay was performed three times by two different analysts on different days and the results were compared. As shown in FIG. 11, the three experiments were similar which is further demonstrated by the low standard deviation on the curve representing the averaged results.

### 1.7 Investigating Stability-Indicating Properties of the Assay

To determine if the GEM formation potency assay is stability-indicating, the rAAVhu68-SMN1 material was subjected to thermal stress at different temperatures for various time periods (Table 3). The genome titers of the samples after thermal stress were determined. Only samples with a small loss in genome titer were tested for potency after their concentrations in the assay were adjusted according to the newly determined genome titers. The results presented in Table 3 demonstrate that there was a significant decrease in potency independent of the loss of genome titer. In conclusion, the GEM formation assay is stability-indicating and can be used for SAR and stability studies.

TABLE 3
Results of the potency assay on rAAVhu68-SMNl thermally-
stressed samples.

| | Control, | 10 min., | 20 min., | 30 min., |
|---|---|---|---|---|
| Test | untreated | 60° C. | 60° C. | 60° C. |
| Genome titer | 2.9E+13 | 2.19E+13 | 1.90E+13 | 1.91E+13 |
| (VG/mL) | | | | |
| Relative | 100 | 74 | 64 | 64 |
| Potency, % | | | | |

### 1.8 Effect of Empty Capsids on the Potency Assay

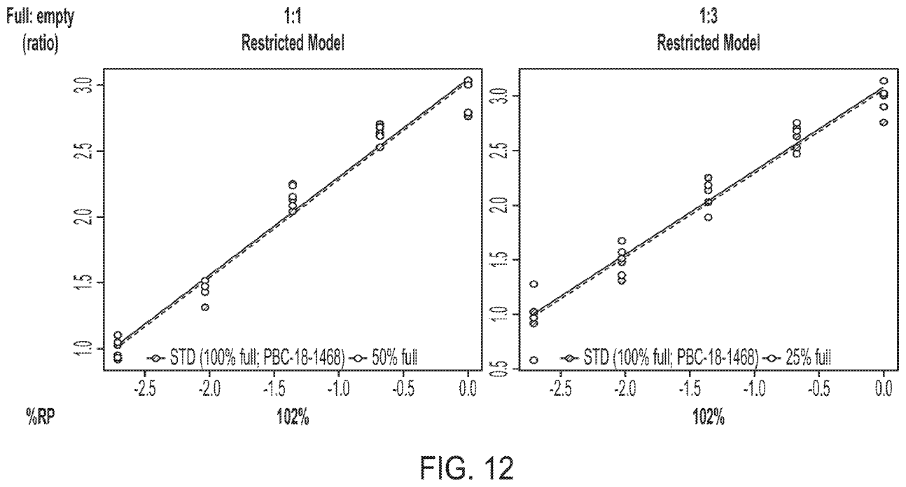

AAV materials contains a fraction of empty AAV particles which can't be eliminated during the purification process and could affect the efficiency of transduction of full particles (e.g., a reference standard for rAAVhu68-SMN1 contains 5.6% of empty particles). To determine the effect of empty particles on viral vector potency, rAAVhu68-SMN1 material containing fixed amount of full particles was spiked with empty capsid preparation to achieve 1:1 and 1:3 ratios between the two. Empty particles were obtained after transient transfection of HEK293 suspension cells with the Helper, Rep/Cap and SMN1 plasmids, separated from full capsids on CIMQ column, neutralized, concentrated and buffer exchanged in Harvard's buffer with 0.01% Pluronic-F68. The capsid titers and the levels of residual impurities in rAAVhu68-SMN1 and empty capsid materials were also determined for calculations of full: empty ratios and matrix evaluation (Table 4). The high levels of HEK HCP found in empty preparation and could potentially affect the infectivity of full particles. However, the empty particle material had no effect on rAAVhu68-SMN1 potency (FIG. 12) and did not produce any activity by itself.

TABLE 4
Results of different tests performed on full and empty
particle materials (NA = not applicable).

| Tests | AAVhu68-SMN1 | Empty particles |
|---|---|---|
| Genome titer | 2.9E13 VG/mL | NA |
| Res. HEK DNA | 4.04E5 pg/mL | 2.42E4 pg/mL |
| Res. Plasmid DNA | 4.62E10 copies/mL | <LLOQ |
| HEK HCP | <LLOQ | 5.08E5 ng/mL |
| AAV9 capsid titer | 5.8E13 VP/mL | 7.84E13 VP/mL |

### CONCLUSION

The cell-based potency assay developed for an AAV-SMN recombinant viral vector, such as rAAVhu68-SMN1, relies on accurate detection and quantification of GEMs by high-content imaging. This assay uses a SH-SY5Y KD neuroblastoma cell line with conditional knock down of SMN1 gene, which decreases background expression of SMN protein. Employment of this cell line makes the assay more relevant to the mechanism of action and improves assay performance. Further, the assay was qualified and demonstrated accuracy of 99.6% with 9.2% precision in the linear range of 50-150% relative potency.

The exemplary assay conditions were as follows:
Cell model: SMN KD Human neuroblastoma SH-SY5YshRNA120 pretreated with doxycyclin.
Cell seeding density: 1×10⁴ cells per well. Plated directly from frozen state.
5-point curves with starting MOI of 4×10⁶ VG/cell and a 1.6-fold serial dilutions are run for standard, sample and control in triplicates.
Helper virus (Ad5) is not necessary.

In conclusion, this assay can be used to measure relative potency of viral vectors containing SMN-encoding gene(s) (e.g., rAAVhu68-SMN1) activity for release, characterization, and stability studies of SMN (e.g., SMN1) drug substance and drug product.

### Example 2: Gemin 2 and SMN1 Co-Localization

The following example demonstrated that Gemin 2 can be visualized by immunofluorescence when host cells were fixed with a 1:1 mix of acetone: methanol. In an effort to improve the staining and the number of GEMS detected, SH-SY5Y KD cells (knockdown with shRNA120) were infected with AAVhu68-SMN1. The goal of this experiment was to demonstrate by CellInsight CX5 High-Content Imaging (HCI) that Gemin 2 and SMN proteins co-localized in the GEMS of SH-SY5Y KD cells.

### Day 0: Plate Cells

1. Frozen SH-SY5Y KD cells pre-treated with doxycycline were thawed for 2 minutes in the water bath at 37° C., then re-suspended in warm medium (42.3% DMEM+42.3% Ham's F-12 Nutrient Mixture+15% FBS).
2. Cells were centrifuged (5 minutes at 250 g), then re-suspended in 5 mL of medium and counted
3. Cells were then plated at 1×10⁴ cells/200 μL or 5×10⁴ cells/mL for 1×10⁶ cells in 20 mL total volume of cells. Doxycycline was added to 0.18×10⁶ viable cells/mL for 91% viable cells (0.9×10⁶ cells; 5 mL cells in 13 mL medium).
4. 200 μL was then added to each well.
5. Each plate was left in the hood for 20 minutes.
6. Each plate was incubated overnight at 37° C. with 5% CO₂.

### Day 1: Cell Infection

1. Media was warmed for 20 minutes in the water bath at 37° C.
2. AAVhu68-SMN1 (4.27e13 GC/ml) was thawed.
3. Dilutions were performed in a 2 mL low-protein binding deep well plate as described below (Table 5), then mixed up and down in each well 6-7 times.

TABLE 5
Dilution of AAV material.

| | Final titer (GC/cells) | AAV material dilution |
|---|---|---|
| A | 2 × 10⁶ | 5 μl of AAV-SMN1 + |
| | | 1595 μl of media |

4. Media was then removed from all the wells containing cells, 150 μl of complete media was added to uninfected cells and 150 μl of the AAVhu68-SMN1.
5. Plates were incubated for 48 hours at 37° C. with 5% CO₂.

### Day 3: Cell Staining

1. Medium was aspirated from plates with a multichannel pipette and dumped directly into bleach.
2. Fixed in 100 μl of 1:1 mix of methanol:acetone (freshly made) for 15 minutes at −20° C.
3. Aspirated fixative and dumped in a bottle in the chemical hood.
4. Washed 2×300 μl with DPBS.
5. Added 300 μl of Licor PBS blocking buffer (take it out of the fridge at the very beginning of the IF protocol), followed by 1.5 hrs and rocked slowly (speed ˜2) on belly dancer at RT.
6. Dumped the blocking buffer in the sink and removed excess of buffer plate facing down on a paper towel.
7. Diluted SMN1 antibody (GTX101047 (rabbit)) and Gemin2 (mouse) antibodies in Licor buffer at 1:250.
8. Added 100 μl of primary antibody (SMN1 and/or Gemin2) per well and rocked slowly (speed ˜2) on belly dancer at RT for 2 hours.
9. Washed 3×300 μl of DPBS+0.1% Tween-20 and rocked slowly (speed ˜2) on belly dancer for a total of 15 minutes (3*5 minutes).
10. All the following steps were performed in the dark (covered plate with aluminum foil paper) to protect the secondary fluorescent antibody from the light.
11. Diluted Alexa-Fluor 594 goat anti-mouse, Alexa-Fluor 488 goat anti-rabbit antibodies 1:500 in Licor blocking buffer.
12. Added 100 μl of the corresponding secondary antibody per wells and rocked slowly (speed ˜2) on belly dancer at RT for 1 hour.
13. Washed 3×300 μl of DPBS+0.1% Tween-20 and rocked slowly (speed ˜2) on belly dancer for a total of 15 minutes. (3*5 minutes).
14. Added 200 μl of DAPI (1:6,000) diluted in DPBS for 20 minutes at RT without agitation.
15. Removed DAPI and add 200 μl of PBS.
16. Conserved at 4° C. in a foil paper.

### Day 4: Imaging

Images were acquired using the CellInsight CX5 HCI Platform.

### Results:

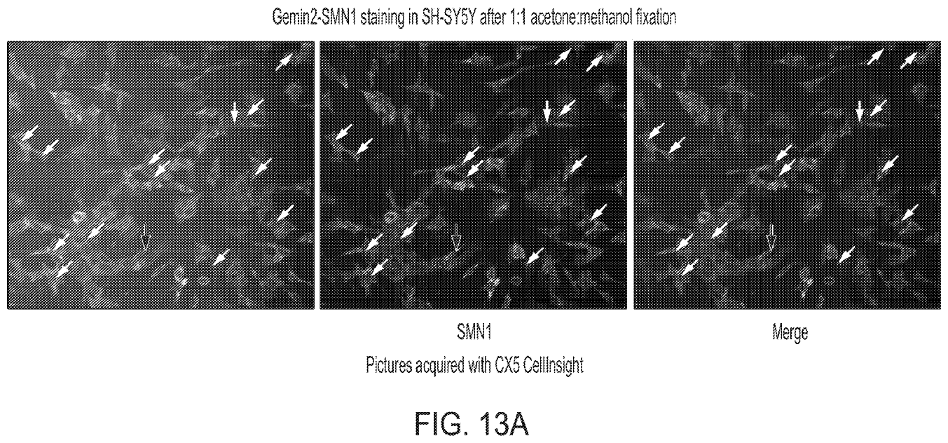

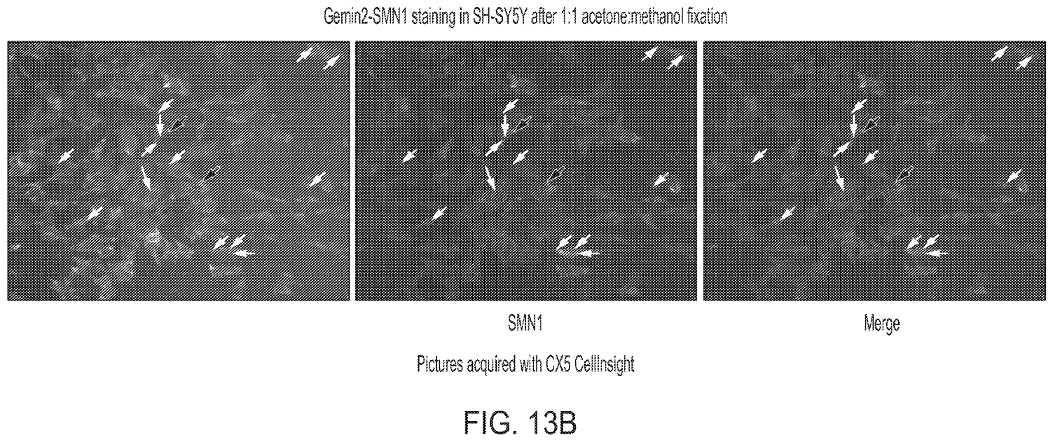

Gemin 2 (red) staining was weak as determined by the high exposure time necessary to acquire a picture (˜0.5s), but Gemin 2 location in the cell was determined. SMN1 (green) staining was good with a low exposure time of ˜0.2s. Very few Gemin 2 positive structures were localized in the cytoplasm (˜1 per pictures, red arrow on the picture above). More SMN1 and Gemin 2-positive structures were visualized after cell infection with AAVhu68-SMN1. The images acquired by HCI show an overlapping signal of SMN1 and Gemin 2 (FIGS. 13A-B).

In conclusion, these results indicate co-localization of SMN1 and Gemin 2 and that the SMN1-structures observed after host cell infection with AAVhu68-SMN1 are GEMs.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

We claim:

1. A method of determining potency of a recombinant viral vector encoding at least one SMN polypeptide comprising:
(a) transducing modified host cells with the recombinant viral vector, wherein the modified host cells comprise decreased expression of the SMN polypeptide relative to an unmodified reference host cell of the same type;
(b) contacting the modified host cells with a first agent for detection of the SMN polypeptide;
(c) contacting the modified host cells with a second agent comprising a detection moiety for detection of the first agent; and
(d) detecting presence of Gemini of coiled bodies (GEMs), thereby determining potency of the at least one SMN polypeptide.

2. The method of claim 1, wherein the recombinant viral vector comprises an adeno-associated viral (AAV) vector, an adenoviral vector, or a retroviral vector.

3. The method of claim 2, wherein the retroviral vector comprises a lentiviral vector or a gammaretroviral vector.

4. The method of claim 2, wherein the AAV vector comprises AAV1, AAV2, AAV3, AAV4, AAV5, AAV6, AAV7, AAV8, AAV9, AAV10, AAV11, or a variant thereof.

5. The method of claim 4, wherein the AAV vector comprises AAVhu68.

6. The method of claim 4 or 5, wherein the AAV vector comprises a SMN1 gene operably linked to a chicken-β actin promoter (CB7).

7. The method of any one of claims 4-6, wherein the AAV vector comprises two ITRs flanking the SMN1 gene.

8. The method of any one of claims 4-7, wherein the AAV vector comprises a rabbit β-globin polyA signal.

9. The method of any one of claims 1-8, wherein the modified host cells comprise a conditional knockdown or a knockout of an SMN1 gene.

10. The method of any one of claims 1-9, wherein the modified host cells comprise at least one shRNA for conditional knockdown of an SMN1 gene.

11. The method of claim 9, wherein the at least one shRNA:
(i) comprises shRNA120 or shRNA 128; and/or
(ii) does not target the recombinant viral vector.

12. The method of any one of claims 1-11, wherein the modified host cells comprise or are mammalian host cells.

13. The method of claim 12, wherein the modified host cells comprise or are human cells.

14. The method of claim 13, wherein the modified host cells comprise or are SH-SY5Y cells.

15. The method of claim 14, wherein the modified host cells comprise or are SH-SY5Y KD cells.

16. The method of any one of claims 1-15, wherein, prior to transduction, one or more of the following occurs:
(i) host cells are frozen and thawed at least once;
(ii) host cells are passaged at least 3 times;
(iii) host cells are treated with doxycycline; and/or
(iv) host cells are seeded at a density of about 5.0×10³ to about 5.0×10⁴ cells/well.

17. The method of any one of claims 1-15, wherein the modified host cells are seeded and transduced within a 24 hour period.

18. The method of any one of claims 1-17, wherein the transduction step (b) is performed at about 5 different MOIs achieved by serial dilution.

19. The method of claim 18, wherein the transduction step (b) is performed with an MOI of about 6.1×10⁵ VG/cell to about 4×10⁶ VG/cell.

20. The method of any one of claims 1-19, wherein a signal to noise ratio is greater than or about 2.5.

21. The method of any one of claims 1-20, wherein the first agent comprises an anti-SMN1 antibody or an antigen-binding fragment thereof or an aptamer.

22. The method of any one of claims 1-21, wherein the detection moiety comprises or is a fluorescent, colorimetric, or enzymatic label.

23. The method of claim 22, wherein the second agent comprises a fluorescently labeled secondary antibody or an antigen-binding fragment thereof.

24. The method of any one of claims 1-23, wherein the presence of GEMs is detected by immunofluorescence.

25. The method of one of claims 1-24, wherein the presence of GEMs is detected by imaging.

26. The method of claim 25, wherein the imaging comprises or is High-Content Imaging (HCI).

27. The method of any one of claims 1-26, wherein the method is performed without or substantially without use of at least one helper function.

28. The method of claim 27, wherein the at least one helper function comprises an Ad2 or Ad5 helper virus.

29. The method of any one of claims 1-28, wherein lower amounts of recombinant viral vector are required for transduction than transducing with an unmodified reference host cells of the same type or a different type.

30. The method of any one of claims 1-29, wherein the method has a low standard deviation.

31. The method of any one of claims 1-30, wherein potency is determined with an accuracy of about 90%, about 91%, about 92%, about 93%, about 94%, about 95%, about 96%, about 97%, about 98%, about 99%, or more.

32. The method of claim 31, wherein potency is determined with a precision of about 5%, about 6%, about 7%, about 8%, about 9%, or about 10%.

33. The method of any one of claims 1-32, wherein the method indicates stability of the recombinant viral vector, e.g., following thermal stress of the recombinant viral vector.

34. The method of any one of claims 1-33, wherein potency of the recombinant viral vector is not affected by presence of empty capsids.

35. The method of any one of claims 1-34, wherein the recombinant viral vector comprises a plurality of empty virus capsids.
